(19) United States
(12) Patent Application Publication
(10) Pub. No.: US 2024/0402160 A1
(43) Pub. Date: Dec. 5, 2024

(54) COMPOSITIONS, DEVICES, AND METHODS OF ECZEMA FOOD SENSITIVITY TESTING

(71) Applicant: Biomerica, Inc., Irvine, CA (US)

(72) Inventor: Zackary Irani-Cohen, Irvine, CA (US)

(73) Assignee: Biomerica, Inc., Irvine, CA (US)

(21) Appl. No.: 18/798,158

(22) Filed: Aug. 8, 2024

Related U.S. Application Data
(63) Continuation of application No. PCT/US2023/062232, filed on Feb. 8, 2023.
(60) Provisional application No. 63/308,000, filed on Feb. 8, 2022.

(51) Int. Cl. G01N 33/543; G01N 33/68

(52) U.S. Cl. CPC G01N 33/54306; G01N 33/6857; G01N 33/6893; G01N 2800/205

(57) ABSTRACT

Contemplated test panels, test kits, and methods using same for food sensitivity in subjects with eczema.

### PRIORITY CLAIM

This application is a continuation of International Application No. PCT/US2023/062232, filed Feb. 8, 2023 and which claims priority to U.S. Provisional Application No. 63/308,000, filed Feb. 8, 2022, each of which are herein incorporated by reference in their entirety.

### FIELD

The field of the disclosure is related to the identification of food items that exacerbate and/or trigger eczema symptomology, particularly as it relates to the testing, identification and possible elimination of certain identified food items in subjects diagnosed with or are suspected to have eczema.

### BACKGROUND

The disclosure includes information that may be useful in understanding the present disclosure. It is not an admission that any of the information provided herein is prior art or relevant to the presently claimed invention, or that any publication specifically or implicitly referenced is prior art.

Eczema is an inflammatory skin condition caused by an over-reactive immune system that results in an inflammatory response leading to dry skin, itchy skin, rashes, scaly patches, blisters and/or skin infections. Eczema affects more than 30 million people in the United States. Moreover, worldwide, about 20% of children and up to 3% of the adult population have some form of eczema. Most typically, eczema is diagnosed by knowledge of the individual's medical history, a careful physical examination, and selected laboratory tests (e.g., patch testing) to rule out other skin diseases and/or identify conditions that accompany eczema. Unfortunately, treatment of eczema is often less than effective and may present new difficulties due to extremely variable individual course.

Food sensitivity, especially as it relates to eczema and the underlying causes of eczema, is not well understood in the medical community. No commercial test specifically for eczema exists. While some commercially available tests for food sensitivity exist, many of these tests suffer from one or more problems, rendering such tests unreliable. Problems associated with these tests include, but are not limited to, high false positive rates, high false negative rates, inter-laboratory variability, the use of an arbitrary selection of foods, and a reliance on a patient's own subjective experiences (i.e., a “trial and error” approach) to identify symptom-causing foods, rendering the tests nearly useless. Unreliable test results can lead to unnecessary and potentially harmful changes in lifestyle, nutritional deficiencies, and/or new symptomology.

No objective, data-driven approach to precisely and reliably identify food items that exacerbate and/or trigger eczema in patients is known. Further, while it is difficult to remove even a single food from all aspects of an individual's diet, in at least some cases, a combination of foods may be contributing to symptomology. As a result, even if a subject shows a positive response to food A, removing food A, from the individual's diet may not relieve symptoms.

Accordingly, there is a need for compositions and methods of food sensitivity testing, especially for the identification and possible elimination of trigger foods for patients identified with or suspected of having eczema, while at the same time, advantageously reducing the number of foods that need to be tested.

### SUMMARY

Described herein are compositions and methods for the identification of food items that exacerbate and/or trigger eczema symptomology in subjects (e.g., human subjects) identified with or are suspected of having eczema, or which alleviate eczema symptomology when removed. One aspect of the disclosure is a test panel for identifying food items that exacerbate and/or trigger eczema symptomology, or which alleviate eczema symptomology when removed, in patients identified with or are suspected of having eczema. In certain embodiments, the test panel includes a plurality of distinct food preparations coupled to a spatially addressable solid carrier.

In another aspect, provided herein is a method of identifying food items that exacerbate and/or trigger eczema symptomology, or which alleviate eczema symptomology when removed, in patients identified with or suspected of having eczema. The method includes a step of contacting a food preparation with a bodily fluid of a patient that is diagnosed with or suspected to have eczema. In some embodiments, the bodily fluid is associated with gender identification. In other embodiments, the bodily fluid is not associated with gender identification. In certain embodiments, the step of contacting is performed under conditions that allow an immunoglobulin (e.g., an antibody, such as an IgG) from the bodily fluid to bind to at least one component of a food preparation. In some embodiments, the method continues with a step of measuring the immunoglobulin (e.g., an antibody, such as an IgG) bound to the at least one component of a food preparation to obtain a signal, and then comparing the signal to a reference value (which in some embodiments may be a gender-stratified reference value) for the food preparation to obtain a result. In some embodiments, the method further includes a step of updating or generating a report using the result.

In another aspect, provided herein is a method of generating a test for identifying of food items that exacerbate and/or trigger eczema symptomology, or which alleviate eczema symptomology when removed, in patients identified with or suspected of having eczema. The method includes a step of obtaining test results for a plurality of distinct food preparations (e.g., a plurality of distinct eczema food preparations). In some embodiments, the test results are based on bodily fluids of patients diagnosed with or suspected of having eczema and bodily fluids of a (healthy) control group not diagnosed with or not suspected of having eczema. In certain embodiments, the method also includes a step of stratifying the test results by gender for each of the distinct food preparations. In other embodiments, the method continues with a step of assigning for a predetermined percentile rank a different cutoff value for male and female patients for each of the distinct food preparations.

In one aspect, the disclosure provides a test panel for testing food sensitivity in a human subject diagnosed with or suspected of having eczema, comprising one or more distinct food preparations, wherein each distinct food preparation is independently coupled to a spatially addressable solid carrier, wherein at least 70% of the one or more distinct food preparations have a raw p-value of ≤0.07 or a false discovery rate (FDR) multiplicity adjusted p-value of ≤0.10.

In one aspect, the disclosure provides a test panel for testing food sensitivity in a human subject diagnosed with or suspected of having eczema, consisting essentially of one or more distinct food preparations, wherein each distinct food preparation is independently coupled to a spatially addressable solid carrier, wherein the one or more distinct food preparations have a raw p-value of ≤0.07 or a false discovery rate (FDR) multiplicity adjusted p-value of ≤0.10. In another aspect, the disclosure provides a test panel for testing food sensitivity in a human subject diagnosed with or suspected of having eczema, consisting of one or more distinct food preparations, wherein each distinct food preparation is independently coupled to a spatially addressable solid carrier, wherein the one or more distinct food preparations have a raw p-value of ≤0.07 or a false discovery rate (FDR) multiplicity adjusted p-value of ≤0.10. In another aspect, the disclosure provides a test panel for testing food sensitivity in a human subject diagnosed with or suspected of having eczema. Each distinct food preparation is independently coupled to a spatially addressable solid carrier and each distinct food preparation has a raw p-value of ≤0.07 or a false discovery rate (FDR) multiplicity adjusted p-value of ≤0.10.

In one embodiment, the one or more distinct food preparations includes at least 4 food preparations selected from foods in Table 1. In another embodiment, the one or more distinct food preparations includes at least 5 food preparations selected from foods in Table 1. In another embodiment, the one or more distinct food preparations includes at least 6 food preparations selected from foods in Table 1. In another embodiment, the one or more distinct food preparations includes at least 8 food preparations selected from foods in Table 1. In another embodiment, the one or more distinct food preparations includes at least 10 food preparations prepared from food items of Table 1. In another embodiment, the one or more distinct food preparations includes at least 12 food preparations prepared from food items of Table 1. In another embodiment, the one or more distinct food preparations only include food preparations selected from the list of food items of Table 1.

In another embodiment, the one or more distinct food preparations has a raw p-value of ≤0.05 or a FDR multiplicity adjusted p-value of ≤0.08. In another embodiment, the one or more distinct food preparations has a raw p-value of ≤0.025 or a FDR multiplicity adjusted p-value of ≤0.07. In another embodiment, at least 75% of the one or more distinct food preparations have a raw p-value of ≤0.07 or FDR multiplicity adjusted p-value of ≤0.10. In another embodiment, at least 80% of the one or more distinct food preparations have a raw p-value of ≤0.07 or FDR multiplicity adjusted p-value of ≤0.10. In another embodiment, at least 85% of the one or more distinct food preparations have a raw p-value of ≤0.07 or FDR multiplicity adjusted p-value of ≤0.10. In another embodiment, at least 90% of the one or more distinct food preparations have a raw p-value of ≤0.07 or FDR multiplicity adjusted p-value of ≤0.10. In another embodiment, at least 95% of the one or more distinct food preparations have a raw p-value of ≤0.07 or FDR multiplicity adjusted p-value of ≤0.10. In another embodiment, each of the one or more distinct food preparations have a raw p-value of ≤0.07 or FDR multiplicity adjusted p-value of ≤0.10.

In another embodiment, FDR multiplicity adjusted p-value is adjusted for age or gender. In another embodiment, FDR multiplicity adjusted p-value is not adjusted for age and gender. In another embodiment, the one or more distinct food preparations comprise crude filtered aqueous extracts. In another embodiment, the one or more distinct food preparations comprise processed aqueous extracts. In another embodiment, the one or more distinct food preparations comprise crude filtered aqueous extracts or processed aqueous extracts. In another embodiment, the p-value is determined by a process comprising comparing assay values of a first subject test cohort that is diagnosed with or is suspected of having eczema with assay values of a second subject test cohort that is not diagnosed with or is not suspected of having eczema. In another embodiment, the p-value is determined by a process comprising comparing a mean immunoglobulin immunoassay score for a distinct food preparation from a first subject test group that is diagnosed with or is suspected of having eczema to at least the 90^{th} percentile rank of a mean or median immunoglobulin immunoassay score for the distinct food preparation from a second subject test group that is not diagnosed with or is not suspected of having eczema. In another embodiment, the p-value is determined by a process comprising comparing a mean or median immunoglobulin immunoassay score for a distinct food preparation from a first subject test group that is diagnosed with or is suspected of having eczema to at least the 95^{th} percentile rank of a mean or median immunoglobulin immunoassay score for the distinct food preparation from a second subject test group that is not diagnosed with or is not suspected of having eczema. In another embodiment, the immunoassay is an ELISA or an antibody capture enzyme immunoassay. In another embodiment, the solid carrier is an array, a multi-well plate, a microtiter plate, a microchip, a bead, a disc, a sensor, an adsorptive film, a membrane, a glass slide, a magnetic particle, a dipstick, or a microfluidic device. In another embodiment, the array is a spotted microarray or a lateral flow array. In another embodiment, the sensor is an electrical sensor, a chemical sensor, or a fiber optic sensor. In another embodiment, the membrane is a nitrocellulose membrane, a polytetrafluorethylene membrane, a cellulose nitrate membrane or a cellulose acetate membrane. In another embodiment, all of the one or more distinct food preparations have an average raw p-value of ≤0.07 or an average false discovery rate (FDR) multiplicity adjusted p-value of ≤0.10. In another embodiment, the FDR multiplicity adjusted p-value is adjusted for age and/or gender.

In another aspect, the disclosure provides a test kit comprising the test panel as disclosed herein.

In another aspect, the disclosure provides an eczema test panel for the simultaneous detection of one or more eczema trigger foods, comprising a plurality of distinct eczema trigger food preparations, wherein each distinct eczema trigger food preparation is independently immobilized to a spatially addressable solid carrier, wherein at least 70% of the plurality of distinct eczema trigger food preparations each have a raw p-value of ≤0.07 or a FDR multiplicity adjusted p-value of ≤0.10.

In another aspect, the disclosure provides an eczema test panel for the simultaneous detection of one or more eczema trigger foods, consisting essentially of, a plurality of distinct eczema trigger food preparations, wherein each distinct eczema trigger food preparation is independently immobilized to a spatially addressable solid carrier, wherein the plurality of distinct eczema trigger food preparations each have a raw p-value of ≤0.07 or a FDR multiplicity adjusted p-value of ≤0.10. In another aspect, the disclosure provides an eczema test panel for the simultaneous detection of one or more eczema trigger foods, consisting of, a plurality of distinct eczema trigger food preparations, wherein each distinct eczema trigger food preparation is independently immobilized to a spatially addressable solid carrier, wherein the plurality of distinct eczema trigger food preparations each have a raw p-value of ≤0.07 or a FDR multiplicity adjusted p-value of ≤0.10.

In some embodiments, the plurality of distinct eczema trigger food preparations includes at least 4 distinct eczema trigger food preparations. In other embodiments, the plurality of distinct eczema trigger food preparations includes at least 5 distinct eczema trigger food preparations. In other embodiments, the plurality of distinct eczema trigger food preparations includes at least 6 distinct eczema trigger food preparations. In other embodiments, the plurality of distinct eczema trigger food preparations includes at least 7 distinct eczema trigger food preparations. In other embodiments, the plurality of distinct eczema trigger food preparations includes at least 8 distinct eczema trigger food preparations. In other embodiments, the plurality of distinct eczema trigger food preparations includes at least 9 distinct eczema trigger food preparations. In other embodiments, the plurality of distinct eczema trigger food preparations includes at least 10 distinct eczema trigger food preparations. In other embodiments, the plurality of distinct eczema trigger food preparations includes at least 11 distinct eczema trigger food preparations. In other embodiments, the plurality of distinct eczema trigger food preparations includes at least 12 distinct eczema trigger food preparations. In other embodiments, each of the plurality of distinct eczema trigger food preparations are distinct eczema trigger food preparations.

In other embodiments, the plurality of distinct eczema trigger food preparations each have a raw p-value of ≤0.05 or FDR multiplicity adjusted p-value of ≤0.08. In other embodiments, the plurality of distinct eczema trigger food preparations each have a raw p-value of ≤0.025 or FDR multiplicity adjusted p-value of ≤0.07. In other embodiments, FDR multiplicity adjusted p-value is adjusted for age or gender. In other embodiments, FDR multiplicity adjusted p-value is adjusted for age and gender. In other embodiments, the plurality of distinct eczema trigger food preparations comprises crude filtered aqueous extracts. In other embodiments, the plurality of distinct eczema trigger food preparations comprises processed aqueous extracts. In other embodiments, the plurality of distinct eczema trigger food preparations comprises crude filtered aqueous extracts or processed aqueous extracts. In other embodiments, the p-value is determined by a process comprising comparing assay values of a first subject test cohort that is diagnosed with or is suspected of having eczema with assay values of a second subject test cohort that is not diagnosed with or is not suspected of having eczema. In other embodiments, the p-value is determined by a process comprising comparing a mean or median immunoglobulin immunoassay scores for a distinct food preparation from a first subject test group that is diagnosed with or is suspected of having eczema to at least the 90^{th} percentile rank of the mean or median immunoglobulin immunoassay scores for the distinct food preparation from a second subject test group that is not diagnosed with or is not suspected of having eczema. In other embodiments, the p-value is determined by a process comprising comparing a mean or median immunoglobulin immunoassay scores for a distinct food preparation from a first subject test group that is diagnosed with or is suspected of having eczema to at least the 95^{th} percentile rank of the mean or median immunoglobulin immunoassay scores for the distinct food preparation from a second subject test group that is not diagnosed with or is not suspected of having eczema. In other embodiments, the immunoassay is an ELISA or an antibody capture enzyme immunoassay. In other embodiments, the solid carrier is an array, a multi-well plate, a microtiter plate, a microchip, a bead, a disc, a sensor, an adsorptive film, a membrane, a glass slide, a magnetic particle, a dipstick, or a microfluidic device. In other embodiments, the array is a spotted microarray or a lateral flow array. In other embodiments, the sensor is an electrical sensor, a chemical sensor, or a fiber optic sensor. In other embodiments, the membrane is a nitrocellulose membrane, a polytetrafluorethylene membrane, a cellulose nitrate membrane or a cellulose acetate membrane. In other embodiments, the totality of the plurality of distinct eczema trigger food preparations has an average raw p-value of ≤0.07 or an average false discovery rate (FDR) multiplicity adjusted p-value of ≤0.10. In other embodiments, the FDR multiplicity adjusted p-value is adjusted for age and/or gender.

In another aspect, the disclosure provides a test kit comprising the eczema test panel as described herein.

In another aspect, the disclosure provides a method of identifying one or more distinct food preparations for a subject known to have or is suspected of having eczema, the method comprising contacting a bodily fluid of a subject that is known to have or is suspected of having eczema with a plurality of distinct food preparations coupled to a solid carrier, wherein the contacting is performed under conditions that allow antibodies from the bodily fluid to bind to a first distinct food preparation, detecting the antibodies bound to the first distinct food preparation to obtain a first immunoassay signal, comparing the first immunoassay signal to a first control signal, and identifying one or more distinct food preparations positive for the subject known to have or is suspected of having eczema.

In another aspect, the disclosure provides a method of identifying a human subject in need of medical treatment for eczema, the method comprising obtaining a bodily fluid from the subject, contacting the bodily fluid of the subject with a plurality of distinct food preparations coupled to a solid carrier, wherein the contacting is performed under conditions that allow antibodies from the bodily fluid to bind to each distinct food preparation in the plurality, detecting the antibodies bound to the each distinct food preparation in the plurality to obtain an immunoassay signal for each distinct food preparation in the plurality, comparing the immunoassay signal for each distinct food preparation in the plurality to a control signal for each distinct food preparation in the plurality, and identifying the distinct food preparations positive for the subject, thereby determining the need for medical treatment for the subject.

In some embodiments, the first control signal is determined by assigning a predetermined percentile rank cutoff value for the first distinct food preparation, and wherein the predetermined percentile rank cutoff value is an at least 90^{th} percentile rank of the control signal from a control group or known standard. In other embodiments, wherein the method further comprises detecting the antibodies bound to a second distinct food preparation to obtain a second immunoassay signal and comparing the second immunoassay signal to a second control signal. In other embodiments, the second control signal is determined by assigning a predetermined percentile rank cutoff value for the second distinct food preparation, and wherein the predetermined percentile rank cutoff value is an at least 90^{th} percentile rank of the second control signal from a control group or known standard.

In other embodiments, the method further comprises detecting the antibodies bound to each of the plurality of distinct food preparations to obtain an immunoassay signal for each distinct food preparation and comparing each immunoassay signal to a control signal for each distinct food preparation. In other embodiments, the control signal for each distinct food preparation is determined by assigning a predetermined percentile rank cutoff value for each distinct food preparation, and wherein the predetermined percentile rank cutoff value is an at least 90^{th} percentile rank of the second control signal from a control group or known standard. In other embodiments, the control signal for each distinct food preparation is determined by assigning a predetermined percentile rank cutoff value for each distinct food preparation, and wherein the predetermined percentile rank cutoff value is an at least 90^{th} percentile rank of the control signal from a control group or known standard. In other embodiments, the detecting comprises measuring or quantifying the antibodies via an immunoassay. In other embodiments, the first control signal is determined by contacting a bodily fluid of a non-eczema healthy control subject with a first distinct food preparation coupled to a solid carrier, wherein the contacting is performed under conditions that allow antibodies from the bodily fluid to bind to at least one component of a distinct food preparation and measuring or quantifying the antibodies via an immunoassay. In other embodiments, the immunoassay is an ELISA or an antibody capture enzyme immunoassay. In other embodiments, the first control signal is determined by comparing the first immunoassay signal against a gender-stratified reference value for the distinct food preparation. In other embodiments, the control signal is determined by comparing the immunoassay signal for each distinct food preparation in the plurality against a gender-stratified reference value for the distinct food preparation. In other embodiments, the subject is diagnosed with eczema. In other embodiments, wherein the method further comprises generating a report for the distinct food preparations positive for the subject. In other embodiments, wherein the method further comprises generating a report for the one or more distinct food preparations positive for the subject for the potential elimination from the subject's diet. In other embodiments, wherein the method further comprises treating the subject having eczema, wherein the treatment comprises eliminating one or more of the identified eczema trigger foods from the subject's diet. In other embodiments, the plurality of distinct food preparations are food preparations having a raw p-value of ≤0.07 or a false discovery rate (FDR) multiplicity adjusted p-value of ≤0.10. In other embodiments, at least 70% of the plurality of distinct food preparations have a raw p-value of ≤0.07 or FDR multiplicity adjusted p-value of ≤0.10. In other embodiments, the totality of the plurality of distinct food preparations has an average raw p-value of ≤0.07 or an average false discovery rate (FDR) multiplicity adjusted p-value of ≤0.10. In other embodiments, the plurality of distinct food preparations are food preparations having a raw p-value of ≤0.05 or FDR multiplicity adjusted p-value of ≤0.08, or a raw p-value of ≤0.025 or FDR multiplicity adjusted p-value of ≤0.07. In other embodiments, the solid carrier includes four or more distinct food preparations. In other embodiments, each distinct food preparation is independently immobilized to the solid carrier in a spatially addressable manner. In other embodiments, the solid carrier is an array, a multi-well plate, a microtiter plate, a microchip, a bead, a disc, a sensor, an adsorptive film, a membrane, a glass slide, a magnetic particle, a dipstick, or a microfluidic device. In other embodiments, the array is a spotted microarray or a lateral flow array. In other embodiments, the sensor is an electrical sensor, a chemical sensor, or a fiber optic sensor. In other embodiments, the membrane is a nitrocellulose membrane, a polytetrafluorethylene membrane, a cellulose nitrate membrane or a cellulose acetate membrane. In other embodiments, the antibody has an isotype selected from the group consisting of IgG, IgA, IgM, and IgD. In other embodiments, the antibody is not an IgE. In other embodiments, the bodily fluid is whole blood, plasma, serum, saliva, urine or a fecal suspension. In other embodiments, FDR multiplicity adjusted p-value is adjusted for age and/or gender. In other embodiments, the one or more distinct food preparations comprises crude filtered aqueous extracts and/or processed aqueous extracts.

In another aspect, the disclosure provides a method of reducing or relieving eczema symptoms in a human subject known to have or suspected of having eczema, the method comprising identifying one or more distinct food preparations that are positive for a subject known to have or is suspected of having eczema according to the method as disclosed herein, wherein eczema symptoms are reduced or alleviated in the subject by eliminating one or more of the identified eczema trigger foods from the subject's diet.

In another aspect, the disclosure provides a method for monitoring the progression of eczema in a human subject, the method comprising contacting a bodily fluid obtained from the subject at a first time point with a plurality of distinct food preparations coupled to a solid carrier, wherein the contacting is performed under conditions that allow antibodies from the bodily fluid to bind to each distinct food preparation in the plurality, detecting the antibodies bound to the each distinct food preparation in the plurality at the first time point to obtain an immunoassay signal for each distinct food preparation in the plurality, contacting a bodily fluid obtained from the subject at a second time point with a plurality of distinct food preparations coupled to a solid carrier, wherein the contacting is performed under conditions that allow antibodies from the bodily fluid to bind to each distinct food preparation in the plurality, detecting the antibodies bound to the each distinct food preparation in the plurality at the second time point to obtain an immunoassay signal for each distinct food preparation in the plurality, comparing the immunoassay signal for each distinct food preparation in the plurality at the first time point and the second time point, wherein an increased amount of at least one immunoassay signal present in the bodily fluid obtained at the second time point compared to the bodily fluid at the first time point indicates progression of eczema.

In another aspect, the disclosure provides a method for monitoring the efficacy of a treatment for eczema in a human subject, the method comprising contacting a bodily fluid obtained from the subject at a first time point with a plurality of distinct food preparations coupled to a solid carrier, wherein the contacting is performed under conditions that allow antibodies from the bodily fluid to bind to each distinct food preparation in the plurality, detecting the antibodies bound to the each distinct food preparation in the plurality at the first time point to obtain an immunoassay signal for each distinct food preparation in the plurality, administering a treatment regimen, contacting a bodily fluid obtained from the subject at a second time point with a plurality of distinct food preparations coupled to a solid carrier, wherein the contacting is performed under conditions that allow antibodies from the bodily fluid to bind to each distinct food preparation in the plurality, detecting the antibodies bound to the each distinct food preparation in the plurality at the second time point to obtain an immunoassay signal for each distinct food preparation in the plurality, comparing the immunoassay signal for each distinct food preparation in the plurality at the first time point and the second time point, wherein a decreased amount of at least immunoassay signal present in the bodily fluid obtained at the second time point compared to the bodily fluid at the first time point is indicative of an effective treatment for eczema.

In certain embodiments, an increased amount of at least 2, at least 3, at least 4, at least 5, at least 6, at least 7, or at least 8 immunoassay signals present in the bodily fluid obtained at the second time point compared to the bodily fluid at the first time point is indicates progression of eczema. In other embodiments, a decreased amount of at least 2, at least 3, at least 4, at least 5, at least 6, at least 7, or at least 8 immunoassay signals present in the bodily fluid obtained at the second time point compared to the bodily fluid at the first time point is indicative of an effective treatment for eczema. In other embodiments, the plurality of distinct food preparations are food preparations having a raw p-value of ≤0.07 or a false discovery rate (FDR) multiplicity adjusted p-value of ≤0.10. In other embodiments, the detecting comprises measuring or quantifying the antibodies via an immunoassay. In other embodiments, the immunoassay is an ELISA or an antibody capture enzyme immunoassay. In other embodiments, wherein the method further comprises treating the subject, wherein the treatment comprises eliminating one or more of the identified distinct food preparations from the subject's diet.

In another aspect, the disclosure provides a method of generating a test panel for food sensitivity in a human subject known to have or suspected of having eczema, the method comprising obtaining immunoassay results for a plurality of distinct eczema trigger food preparations, wherein the immunoassay results are based on bodily fluids of subjects known to have or suspected of having eczema and bodily fluids of a control group not known to have or not suspected to have eczema, selecting a plurality of the distinct food preparations, wherein the distinct food preparations are food preparations having a raw p-value of ≤0.07 or a false discovery rate (FDR) multiplicity adjusted p-value of ≤0.10, and generating a test comprising of the selected distinct food preparations.

In some embodiments, the method further comprises assigning a predetermined percentile rank cutoff value for each of the plurality of the distinct food preparations, wherein the predetermined percentile rank cutoff value is determined using an at least 90^{th} percentile rank of the immunoassay results based on bodily fluids of the control group. In other embodiments, the method further comprises comparing the immunoassay results for the plurality of distinct food preparations based on bodily fluids of patients known to have or suspected of having eczema to the predetermined percentile rank cutoff value. In some embodiments, the method further comprising stratifying the immunoassay results by gender for each of the distinct food preparations. In some embodiments, the method further comprising assigning a predetermined percentile rank a cutoff value for male and female subjects for each of the distinct food preparations. In some embodiments, the method further comprising normalizing the immunoassay results to the patient's total IgG. In some embodiments, the method further comprising normalizing the immunoassay results to the global mean of the patient's food specific IgG results. In some embodiments, the predetermined percentile rank is an at least 95^{th} percentile rank. In some embodiments, the plurality of distinct food preparations are food preparations having a raw p-value of ≤0.07 or a false discovery rate (FDR) multiplicity adjusted p-value of ≤0.10. In some embodiments, the totality of the plurality of distinct food preparations has an average raw p-value of ≤0.07 or an average false discovery rate (FDR) multiplicity adjusted p-value of ≤0.10. In some embodiments, the plurality of distinct food preparations are food preparations having a raw p-value of ≤0.05 or FDR multiplicity adjusted p-value of ≤0.08, or a raw p-value of ≤0.025 or FDR multiplicity adjusted p-value of ≤0.07. In some embodiments, the plurality of food preparations is coupled to the solid carrier in a spatially addressable manner. In some embodiments, the solid carrier includes four or more food preparations. In some embodiments, the solid carrier is an array, a multi-well plate, a microtiter plate, a microchip, a bead, a disc, a sensor, an adsorptive film, a membrane, a glass slide, a magnetic particle, a dipstick, or a microfluidic device. In some embodiments, the array is a spotted microarray or a lateral flow array. In some embodiments, the sensor is an electrical sensor, a chemical sensor, or a fiber optic sensor. In some embodiments, the membrane is a nitrocellulose membrane, a polytetrafluorethylene membrane, a cellulose nitrate membrane or a cellulose acetate membrane. In some embodiments, at least 70% of the distinct food preparations have a raw p-value of ≤0.07 or FDR multiplicity adjusted p-value of ≤0.10. In some embodiments, at least 80% of the distinct food preparations have a raw p-value of ≤0.07 or FDR multiplicity adjusted p-value of ≤0.10. In some embodiments, at least 90% of the distinct food preparations have a raw p-value of ≤0.07 or FDR multiplicity adjusted p-value of ≤0.10. In some embodiments, at least 95% of the distinct food preparations have a raw p-value of ≤0.07 or FDR multiplicity adjusted p-value of ≤0.10. In some embodiments, FDR multiplicity adjusted p-value is adjusted for age and/or gender. In some embodiments, the distinct food preparations comprise crude filtered aqueous extracts and/or processed aqueous extracts.

In another aspect, the disclosure provides a test panel generated according to the methods described herein.

In another aspect, the disclosure provides a test kit comprising the test panel according to the methods described herein.

### DETAILED DESCRIPTION

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

The inventors have discovered that food preparations for use in food tests to identify foods that exacerbate and/or trigger symptomology in patients diagnosed with or suspected of having eczema not equally well predictive and/or associated with eczema. Indeed, various experiments disclosed herein have revealed that among a wide variety of food items, certain food items are highly indicative of/associated with eczema symptomology whereas others have no statistically significant association with eczema symptomology.

Even more unexpectedly, the inventors discovered that in addition to the high variability of food items, gender variability with respect to response in a test plays a substantial role in the determination of association of a food item with eczema. Consequently, based on the inventors' findings and further contemplations, compositions and methods of using same are now presented with substantially higher predictive power in the identification of food items that could be eliminated for reduction of eczema signs and symptoms.

The present disclosure provides many exemplary embodiments of the inventive subject matter. Although certain embodiments represent a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

In some embodiments, the numbers expressing quantities or ranges, used to describe and claim certain embodiments of the disclosure are to be understood as being modified in some instances by the term “about.” Accordingly, in some embodiments, the numerical parameters set forth in the written description and claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclosure may contain certain errors necessarily resulting from the standard deviation found in their respective testing measurements. Unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

The terms “comprising,” “consisting essentially of,” and “consisting of” define the scope of a claim with respect to what unrecited additional components or steps, if any, are excluded from the scope of the claim. Only the transitional phrases “consisting of” and “consisting essentially of” are closed or semi-closed transitional phrases, respectively. It should be understood that, in general, where the invention, or aspects of the invention, is/are referred to as comprising particular elements, features, etc., certain embodiments of the invention or aspects of the invention consist, or consist essentially of, such elements, features, etc.

The term “average discriminatory p-value”, as used herein, generally refers to an average of all the p-values with a particular probability (e.g., ≤0.07) as determined by raw p-value or with a particular probability (e.g., ≤0.1) as determined by FDR multiplicity adjusted p-value that were identified by analytical methods described herein. In one embodiment, average discriminatory p-value refers to an average of all the p-values with a particular probability (e.g., ≤0.07) as determined by raw p-value or with a particular probability (e.g., ≤0.1) as determined by FDR multiplicity adjusted p-value that were identified by analytical methods described herein. In another embodiment, average discriminatory p-value refers to an average of all the p-values that are ≤0.07 as determined by raw p-value that were identified by analytical methods described herein. In another embodiment, average discriminatory p-value refers to an average of all the p-values that are ≤0.065 as determined by raw p-value that were identified by analytical methods described herein. In another embodiment, average discriminatory p-value refers to an average of all the p-values that are ≤0.05 as determined by raw p-value that were identified by analytical methods described herein. In another embodiment, average discriminatory p-value refers to an average of all the p-values that are ≤0.1 as determined by False Discovery Rate (FDR) multiplicity adjusted p-value that were identified by analytical methods described herein. In another embodiment, average discriminatory p-value refers to an average of all the p-values that are ≤0.09 as determined by False Discovery Rate (FDR) multiplicity adjusted p-value that were identified by analytical methods described herein. In another embodiment, average discriminatory p-value refers to an average of all the p-values that are ≤0.095 as determined by False Discovery Rate (FDR) multiplicity adjusted p-value that were identified by analytical methods described herein. In another embodiment, average discriminatory p-value refers to an average of all the p-values that are ≤0.09 as determined by False Discovery Rate (FDR) multiplicity adjusted p-value that were identified by analytical methods described herein. In another embodiment, average discriminatory p-value refers to an average of all the p-values that are ≤0.085 as determined by False Discovery Rate (FDR) multiplicity adjusted p-value that were identified by analytical methods described herein. In another embodiment, average discriminatory p-value refers to an average of all the p-values that are ≤0.08 as determined by False Discovery Rate (FDR) multiplicity adjusted p-value that were identified by analytical methods described herein. In another embodiment, average discriminatory p-value refers to an average of all the p-values that are ≤0.075 as determined by False Discovery Rate (FDR) multiplicity adjusted p-value that were identified by analytical methods described herein. In another embodiment, average discriminatory p-value refers to an average of all the p-values that are ≤0.07 as determined by False Discovery Rate (FDR) multiplicity adjusted p-value that were identified by analytical methods described herein. In another embodiment, average discriminatory p-value refers to an average of all the p-values that are ≤0.065 as determined by False Discovery Rate (FDR) multiplicity adjusted p-value that were identified by analytical methods described herein. In another embodiment, average discriminatory p-value refers to an average of all the p-values that are ≤0.06 as determined by False Discovery Rate (FDR) multiplicity adjusted p-value that were identified by analytical methods described herein.

The term “food preparation”, as used herein, refers to a solubilized aqueous extraction of a specific food item. In certain embodiments, the specific food item is solubilized using a blender or similar apparatus, in the presence of a buffer and the food item is processed until the structure of the food item is broken down into a homogenous liquid suspension or solution.

The term “one component of the food preparation” or “a component of the food preparation”, as used herein, refers to any portion of a food preparation (e.g., but not limited to, a protein(s), a lipid(s), a sugar(s), etc., or combination thereof) that is capable of being bound by an immunoglobulin and/or that is antigenic (i.e., capable of inducing and/or eliciting an immune response in a subject or patient).

The term “spatially addressable” or “individually addressable,” as used herein, refers to a manner of arranging a food preparation (or a plurality of food preparations) on a solid carrier (e.g. an array, as ELISA well, a spotted microarray, etc.), such that said food preparation (or said plurality of food preparations) is immobilized (e.g., covalently coupled, etc.) to the solid carrier in a manner that sufficiently separates said food preparation from other food preparations immobilized to the solid carrier, and that sufficiently allows for the detection of an immunoglobulin (e.g., an IgG or other binding molecule) capable of binding to said food preparation (or a component thereof).

The term “subject” or “patient,” as used interchangeably, refers to either a human or a non-human animal. In one embodiment, a subject is a human. In another embodiment, a subject is a non-human animal.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

The term “trigger food”, as used herein, broadly refers to a food preparation, or a component thereof, which results in a significantly elevated response (e.g., an increase in the level of an antibody) in a subject exposed to the food preparation, or a component thereof, wherein the elevated response is highly correlated to the presence of a disease/syndrome symptom(s), and which potentially may trigger and/or exacerbate a disease/syndrome symptom(s), and/or which may potentially result in an alleviation in symptoms when consumed and/or which may potentially result in a reduction of symptoms when not consumed. In one embodiment, a trigger food may be characterized by a raw p-value of about ≤0.15. In another embodiment, a trigger food may be characterized by a raw p-value of about ≤0.10. In another embodiment, a trigger food may be characterized by a raw p-value of about ≤0.075. In another embodiment, a trigger food may be characterized by a raw p-value of about ≤0.05. In another embodiment, a trigger food may be characterized by a raw p-value of about ≤0.025. In another embodiment, a trigger food may be characterized by a raw p-value of about ≤0.020. In other embodiments, a trigger food may be characterized by a False Discovery Rate (FDR) multiplicity adjusted p-value of about ≤0.10. In another embodiment, a trigger food may be characterized by a False Discovery Rate (FDR) multiplicity adjusted of about ≤0.09. In another embodiment, a trigger food may be characterized by a False Discovery Rate (FDR) multiplicity adjusted p-value of about ≤0.08. In yet another embodiment, a trigger food may be characterized by a False Discovery Rate (FDR) multiplicity adjusted p-value of about ≤0.07.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope of any embodiments of the disclosure otherwise claimed. No language in the specification should be construed as indicating any non-claimed element essential to the practice of any embodiments of the disclosure.

Groupings or listings of alternative elements or embodiments of the disclosure disclosed herein are not to be construed as limitations. Where elements are presented as groups or lists, (e.g., in Markush group or similar format) it is to be understood that each subgroup of the elements is also disclosed, and any element(s) can be removed from the group or list. One or more members of a group or list can be included in, or deleted from, a group for reasons of convenience and/or patentability. When any such inclusion or deletion occurs, the specification is herein deemed to contain the group as modified thus fulfilling the written description of all Markush groups used in the appended claims.

Methods and Compositions Relating to the Identification of Food Items that Exacerbate and/or Trigger Eczema Symptomology

In one aspect, the present disclosure therefore contemplates a test panel, test kit, array, etc. that is suitable for identifying food items that exacerbate and/or trigger eczema symptomology, or which alleviate eczema symptomology when removed from a subject's diet (i.e., not consumed), in subjects where the subject is diagnosed with or is suspected of having eczema. In some embodiments, the test panel, test kit, array, etc., will include one or more (e.g., a plurality) of distinct food preparations (e.g., raw or processed extract(s), e.g., an aqueous extract with optional co-solvent, which may or may not be filtered)) that are coupled to individually addressable respective solid carriers (e.g., in the form of an array or a micro well plate), wherein the distinct food preparations have a raw p-value of ≤0.07 or a FDR multiplicity adjusted p-value of ≤0.10. In certain embodiments, the distinct food preparations have an average raw p-value of ≤0.07 or an average FDR multiplicity adjusted p-value of ≤0.10. In certain embodiments, the plurality of distinct food preparations consists essentially of food preparations that each has a raw p-value of ≤0.07 or a FDR multiplicity adjusted p-value of ≤0.10. In other embodiments, at least 70% (or at least 75%, or at least 80%, or at least 85%, or at least 90%, or at least 95%) of the plurality of distinct food preparations has a discriminatory p-value of ≤0.07 as determined by raw p-value or a discriminatory p-value of ≤0.10 as determined by FDR multiplicity adjusted p-value. In preferred embodiments, the test panel, test kit, array, etc., is for use in the simultaneous detection of one or more eczema trigger foods.

In some embodiments, the numbers expressing quantities of ingredients, properties such as concentration, reaction conditions, and so forth, used to describe and claim certain embodiments of the disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the disclosure are approximations, the numerical values set forth in the specific examples are reported as precisely as practicable. The numerical values presented in some embodiments of the disclosure may contain certain errors necessarily resulting from the standard deviation found in their respective testing measurements. Moreover, and unless the context dictates the contrary, all ranges set forth herein should be interpreted as being inclusive of their endpoints and open-ended ranges should be interpreted to include only commercially practical values. Similarly, all lists of values should be considered as inclusive of intermediate values unless the context indicates the contrary.

While not limiting to the inventive subject matter, food preparations will in certain embodiments be drawn from foods generally known or suspected to exacerbate and/or trigger signs or symptoms of eczema when consumed, or which alleviate eczema symptomology when removed from a subject's diet (i.e., not consumed). Particularly suitable food preparations may be identified by the experimental methods disclosed herein. Thus, it should be appreciated that the food items (e.g., trigger foods) need not be limited to the items described herein, but that all items are contemplated that can be identified by the methods presented herein. Therefore, in certain embodiments, exemplary food preparations include at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty or more food preparations selected from foods of Table 1.

Using bodily fluids from patients diagnosed with or suspected of having eczema and healthy control group individuals (i.e., those not diagnosed with or not suspected of having eczema), numerous additional food items (i.e., trigger foods) may be identified as described herein. In one embodiment, identified food items (i.e., trigger foods) will have high discriminatory power and as such have a raw p-value of ≤0.15. In other embodiments, identified food items (i.e., trigger foods) will have high discriminatory power and as such have a raw p-value of ≤0.14. In other embodiments, identified food items (i.e., trigger foods) will have high discriminatory power and as such have a raw p-value of ≤0.13. In other embodiments, identified food items (i.e., trigger foods) will have high discriminatory power and as such have a raw p-value of ≤0.12. In other embodiments, identified food items (i.e., trigger foods) will have high discriminatory power and as such have a raw p-value of ≤0.11. In other embodiments, identified food items (i.e., trigger foods) will have high discriminatory power and as such have a raw p-value of ≤0.10. In other embodiments, identified food items (i.e., trigger foods) will have high discriminatory power and as such have a raw p-value of ≤0.09. In other embodiments, identified food items (i.e., trigger foods) will have high discriminatory power and as such have a raw p-value of ≤0.08. In other embodiments, identified food items (i.e., trigger foods) will have high discriminatory power and as such have a raw p-value of ≤0.07. In other embodiments, identified food items (i.e., trigger foods) will have high discriminatory power and as such have a raw p-value of ≤0.06. In yet other embodiments, identified food items (i.e., trigger foods) will have high discriminatory power and as such have a raw p-value of ≤0.05. In certain embodiments, identified food items (i.e., trigger foods) will have high discriminatory power and as such have a False Discovery Rate (FDR) multiplicity adjusted p-value of ≤0.20. In certain embodiments, identified food items (i.e., trigger foods) will have high discriminatory power and as such have a False Discovery Rate (FDR) multiplicity adjusted p-value of ≤0.19. In certain embodiments, identified food items (i.e., trigger foods) will have high discriminatory power and as such have a False Discovery Rate (FDR) multiplicity adjusted p-value of ≤0.18. In certain embodiments, identified food items (i.e., trigger foods) will have high discriminatory power and as such have a False Discovery Rate (FDR) multiplicity adjusted p-value of ≤0.17. In certain embodiments, identified food items (i.e., trigger foods) will have high discriminatory power and as such have a False Discovery Rate (FDR) multiplicity adjusted p-value of ≤0.16. In certain embodiments, identified food items (i.e., trigger foods) will have high discriminatory power and as such have a False Discovery Rate (FDR) multiplicity adjusted p-value of ≤0.15. In certain embodiments, identified food items (i.e., trigger foods) will have high discriminatory power and as such have a False Discovery Rate (FDR) multiplicity adjusted p-value of ≤0.14. In certain embodiments, identified food items (i.e., trigger foods) will have high discriminatory power and as such have a False Discovery Rate (FDR) multiplicity adjusted p-value of ≤0.13. In certain embodiments, identified food items (i.e., trigger foods) will have high discriminatory power and as such have a False Discovery Rate (FDR) multiplicity adjusted p-value of ≤0.12. In certain embodiments, identified food items (i.e., trigger foods) will have high discriminatory power and as such have a False Discovery Rate (FDR) multiplicity adjusted p-value of ≤0.11. In certain embodiments, identified food items (i.e., trigger foods) will have high discriminatory power and as such have a False Discovery Rate (FDR) multiplicity adjusted p-value of ≤0.10. In other embodiments, identified food items (i.e., trigger foods) will have high discriminatory power and as such have a False Discovery Rate (FDR) multiplicity adjusted p-value of ≤0.09. In other embodiments, identified food items (i.e., trigger foods) will have high discriminatory power and as such have a False Discovery Rate (FDR) multiplicity adjusted p-value of ≤0.08. In other embodiments, identified food items (i.e., trigger foods) will have high discriminatory power and as such have a False Discovery Rate (FDR) multiplicity adjusted p-value of ≤0.07. In other embodiments, identified food items (i.e., trigger foods) will have high discriminatory power and as such have a False Discovery Rate (FDR) multiplicity adjusted p-value of ≤0.06. In other embodiments, identified food items (i.e., trigger foods) will have high discriminatory power and as such have a False Discovery Rate (FDR) multiplicity adjusted p-value of ≤0.05. In other embodiments, identified food items (i.e., trigger foods) will have high discriminatory power and as such have a False Discovery Rate (FDR) multiplicity adjusted p-value of ≤0.04. In other embodiments, identified food items (i.e., trigger foods) will have high discriminatory power and as such have a False Discovery Rate (FDR) multiplicity adjusted p-value of ≤0.03. In other embodiments, identified food items (i.e., trigger foods) will have high discriminatory power and as such have a False Discovery Rate (FDR) multiplicity adjusted p-value of ≤0.02. In other embodiments, identified food items (i.e., trigger foods) will have high discriminatory power and as such have a False Discovery Rate (FDR) multiplicity adjusted p-value of ≤0.01. In other embodiments, identified food items (i.e., trigger foods) will have high discriminatory power and as such have a False Discovery Rate (FDR) multiplicity adjusted p-value of ≤0.0075. In other embodiments, identified food items (i.e., trigger foods) will have high discriminatory power and as such have a False Discovery Rate (FDR) multiplicity adjusted p-value of ≤0.005. In other embodiments, identified food items (i.e., trigger foods) will have high discriminatory power and as such have a False Discovery Rate (FDR) multiplicity adjusted p-value of ≤0.0025. In other embodiments, identified food items (i.e., trigger foods) will have high discriminatory power and as such have a False Discovery Rate (FDR) multiplicity adjusted p-value of ≤0.001.

In certain embodiments, identified food preparations will have high discriminatory power and, as such, will have a raw p-value of ≤0.15, ≤0.10, ≤0.075, or ≤0.05, and/or a False Discovery Rate (FDR) multiplicity adjusted p-value of ≤0.10, ≤0.09, ≤0.08, or ≤0.07. In one embodiment, identified food preparations will have high discriminatory power and as such have a raw p-value of ≤0.15. In other embodiments, identified food preparations will have high discriminatory power and as such have a raw p-value of ≤0.14. In other embodiments, identified food preparations will have high discriminatory power and as such have a raw p-value of ≤0.13. In other embodiments, identified food preparations will have high discriminatory power and as such have a raw p-value of ≤0.12. In other embodiments, identified food preparations will have high discriminatory power and as such have a raw p-value of ≤0.11. In other embodiments, identified food preparations will have high discriminatory power and as such have a raw p-value of ≤0.10. In other embodiments, identified food preparations will have high discriminatory power and as such have a raw p-value of ≤0.09. In other embodiments, identified food preparations will have high discriminatory power and as such have a raw p-value of ≤0.08. In other embodiments, identified food preparations will have high discriminatory power and as such have a raw p-value of ≤0.07. In other embodiments, identified food preparations will have high discriminatory power and as such have a raw p-value of ≤0.06. In yet other embodiments, identified food preparations will have high discriminatory power and as such have a raw p-value of ≤0.05. In certain embodiments, identified food preparations will have high discriminatory power and as such have a False Discovery Rate (FDR) multiplicity adjusted p-value of ≤0.10. In other embodiments, identified food preparations will have high discriminatory power and as such have a False Discovery Rate (FDR) multiplicity adjusted p-value of ≤0.09. In other embodiments, identified food preparations will have high discriminatory power and as such have a False Discovery Rate (FDR) multiplicity adjusted p-value of ≤0.08. In yet other embodiments, identified food preparations will have high discriminatory power and as such have a False Discovery Rate (FDR) multiplicity adjusted p-value of ≤0.07.

Therefore, where a test panel, test kit, array, etc. has multiple (i.e., a plurality of) food preparations, it is contemplated that in certain embodiments, the plurality of distinct food preparations has an average raw p-value of ≤0.07 or an average False Discovery Rate (FDR) multiplicity adjusted p-value of ≤0.10. In other embodiments, the plurality of distinct food preparations has an average raw p-value of ≤0.05 or an average False Discovery Rate (FDR) multiplicity adjusted p-value of ≤0.08. In other embodiments, the plurality of distinct food preparations has an average raw p-value of ≤0.025 or an average False Discovery Rate (FDR) multiplicity adjusted p-value of ≤0.07. In other embodiments, it should be appreciated that in certain embodiments, the FDR multiplicity adjusted p-value may be adjusted for at least one of age and gender, while in yet other embodiments, the FDR multiplicity adjusted p-value may be adjusted for both age and gender. On the other hand, where a test panel, test kit, array, etc. is stratified for use with a single gender, it is also contemplated that in a test panel, test kit, etc., at least 70%, or at last 75%, or at least 80%, or at least 85%, or at least 90%, or at least 95%, or all of the plurality of distinct food preparations, when adjusted for a single gender, have an average raw p-value of ≤0.07 or an average FDR multiplicity adjusted p-value of ≤0.10. Furthermore, it should be appreciated that other stratifications (e.g., dietary preference, ethnicity, place of residence, genetic predisposition or family history, etc.) are also contemplated.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate certain embodiments of the disclosure and does not pose a limitation on the scope of the any embodiments of the disclosure otherwise claimed. No language in the specification should be construed as indicating any non-claimed element essential to the practice of the disclosure.

Of course, it should be noted that the particular format of the test panel may vary considerably. Consequently, the solid carrier to which the food preparations are coupled may include an array (e.g., a spotted microarray or a lateral flow array), a multiwell plate, a microtiter plate, a microchip, a bead (e.g., color-coded bead or magnetic bead), a disc, a sensor (e.g., an electrical sensor, a chemical sensor, or a fiber optic sensor), an adsorptive film (e.g., nitrocellulose or micro/nanoporous polymeric film), a membrane (e.g., nitrocellulose membrane, a polytetrafluorethylene membrane, a cellulose nitrate membrane or a cellulose acetate membrane), a glass slide, a magnetic particle, a dipstick, or a microfluidic device (e.g., a printed copper sensor or microchip).

In another aspect, the disclosure contemplates a method of identifying food items that trigger and/or exacerbate eczema symptomology in subjects (e.g., human subjects) known to have or suspected of having eczema. In certain embodiments, such methods will include a step of contacting a bodily fluid (e.g., including, but not limited to, whole blood, plasma, serum, saliva, urine, or a fecal suspension) of a subject that is known to have or is suspected of having eczema with a plurality of distinct eczema trigger food preparations. The step of contacting is performed, in certain embodiments, under conditions that allow an immunoglobulin (antibody), e.g., IgG (or IgA or IgM or IgD), from the bodily fluid to bind to at least one component of a food preparation of the plurality of distinct eczema trigger food preparations, wherein the immunoglobulin (antibody) bound to the component(s) of the food preparation are then quantified/measured to obtain a signal (e.g., an immunoassay signal). In some embodiments, the signal is then compared against a gender-stratified reference value (e.g., at least a 90th percentile value) for the food preparation using the gender identification to obtain a result, which is then used to update or generate a report (e.g., written medical report; oral report of results from doctor to patient; written or oral directive from physician based on results). In another embodiment, the method includes the step of detecting the antibodies bound to the distinct food preparations to obtain an immunoassay signal. In another embodiment, the method includes the step of comparing the immunoassay signal to a first control signal. In another embodiment, the immunoglobulin (antibody) is not an IgE. In another embodiment, the immunoglobulin (antibody) is not an IgA. In another embodiment, the immunoglobulin (antibody) is not an IgM. In another embodiment, the immunoglobulin (antibody) is not an IgD.

In another aspect, the disclosure contemplates a method of identifying a human subject in need of medical treatment for eczema. In certain embodiments, such methods will include a step of obtaining a bodily fluid from the subject and contacting a bodily fluid (e.g., including, but not limited to, whole blood, plasma, serum, saliva, urine, or a fecal suspension) of a subject that is known to have or is suspected of having eczema with a plurality of distinct food preparations (e.g., eczema trigger food preparations). The step of contacting is performed, in certain embodiments, under conditions that allow an immunoglobulin (antibody), e.g., IgG (or IgA or IgM or IgD), from the bodily fluid to bind to at least one component of a food preparation of the plurality of distinct food preparations (e.g., eczema trigger food preparations), wherein the immunoglobulin (antibody) bound to the component(s) of the food preparation are then quantified/measured to obtain a signal (e.g., an immunoassay signal). In some embodiments, the signal is then compared against a gender-stratified reference value (e.g., at least a 90th percentile value) for the food preparation using the gender identification to obtain a result, which is then used to update or generate a report (e.g., written medical report; oral report of results from doctor to patient; written or oral directive from physician based on results). In another embodiment, the method includes the step of detecting the antibodies bound to the each distinct food preparation (e.g., eczema trigger food preparations) in the plurality to obtain an immunoassay signal for each distinct eczema trigger food preparation in the plurality. In another embodiment, the immunoglobulin (antibody) is not an IgE. In another embodiment, the immunoglobulin (antibody) is not an IgA. In another embodiment, the immunoglobulin (antibody) is not an IgM. In another embodiment, the immunoglobulin (antibody) is not an IgD.

In preferred embodiments, such methods will not be limited to a single food preparation, but will employ multiple different food preparations (i.e., a plurality of distinct food preparations). As noted herein, suitable food preparations can be identified using various methods as described herein. As also noted herein, in some embodiments, at least some, or all of the different food preparations have an average raw p-value of ≤0.07 (or ≤0.065, or ≤0.06, or ≤0.055, or ≤0.05, or ≤0.045, or ≤0.04, or ≤0.035, or ≤0.03, or ≤0.025), and/or a FDR multiplicity adjusted p-value of ≤0.10 (or ≤0.095, or ≤0.09, or ≤0.085, or ≤0.08, or ≤0.075, or ≤0.07).

While in certain embodiments food preparations are prepared from single food items as crude extracts, or crude filtered extracts, it is contemplated that food preparations can be prepared from mixtures of a plurality of food items (e.g., a mixture of citrus comprising lemon, orange, and a grapefruit, a mixture of rice comprising a brown rice and white rice, a mixture of sugars comprising honey, malt, and cane sugar. In some embodiments, it is also contemplated that food preparations can be prepared from purified food antigens or recombinant food antigens.

As provided in certain embodiments, the food preparation is immobilized on a solid surface (typically in a spatially addressable manner), accordingly, it is contemplated that the step of measuring the immunoglobulin (e.g., IgG or other type of antibody) bound to the component of the food preparation is performed via an immunoassay test (e.g., an ELISA or antibody capture enzyme immunoassay). Exemplary solid surfaces include, but are not limited to, a multiwell plate, such that each food preparation may be isolated to a separate microwell. In other embodiments, the solid surface is an array (e.g., a spotted microarray or a lateral flow array), a multiwell plate, a microtiter plate, a microchip, a bead (e.g., color-coded bead or magnetic bead), a disc, a sensor (e.g., an electrical sensor, a chemical sensor, or a fiber optic sensor), an adsorptive film (e.g., nitrocellulose or micro/nanoporous polymeric film), a membrane (e.g., nitrocellulose membrane, a polytetrafluorethylene membrane, a cellulose nitrate membrane or a cellulose acetate membrane), a glass slide, a magnetic particle, a dipstick, or a microfluidic device, (e.g., a printed copper sensor or microchip). In certain embodiments, the food preparation will be coupled to, or immobilized on, the solid surface. In other embodiments, the food preparation(s) will be coupled to a molecular tag that allows for binding to human immunoglobulins (e.g., IgG) in solution.

In another aspect, the disclosure contemplates a method of reducing or relieving eczema symptoms in a human subject known to have or suspected of having eczema, the method by identifying one or more eczema trigger foods that are positive for a subject known to have or is suspected of having eczema as described herein, wherein eczema symptoms are reduced or alleviated in the subject by eliminating one or more of the identified eczema trigger foods from the subject's diet.

In another aspect, the disclosure contemplates a method for monitoring the progression of eczema in a human subject. In certain embodiments, such method includes a step of contacting a bodily fluid obtained from the subject at a first time point with a plurality of distinct food preparations (e.g., eczema trigger food preparations) coupled to a solid carrier, wherein the contacting is performed under conditions that allow antibodies from the bodily fluid to bind to each distinct food preparation (e.g., eczema trigger food preparation) in the plurality. In other embodiments, the method includes a step of detecting the antibodies bound to the each distinct food preparation (e.g., eczema trigger food preparation) in the plurality at the first time point to obtain an immunoassay signal for each distinct food preparation (e.g., eczema trigger food preparation) in the plurality. In other embodiments, the method includes a step of contacting a bodily fluid obtained from the subject at a second time point with a plurality of distinct food preparations (e.g., eczema trigger food preparations) coupled to a solid carrier, wherein the contacting is performed under conditions that allow antibodies from the bodily fluid to bind to each distinct food preparation (e.g., eczema trigger food preparation) in the plurality. In other embodiments, the method includes a step of detecting the antibodies bound to the each distinct food preparation (e.g., eczema trigger food preparation) in the plurality at the second time point to obtain an immunoassay signal for each distinct food preparation (e.g., eczema trigger food preparation) in the plurality. In other embodiments, the method includes a step of comparing the immunoassay signal for each distinct food preparation (e.g., eczema trigger food preparation) in the plurality at the first time point and the second time point, wherein an increased amount of at least one immunoassay signal present in the bodily fluid obtained at the second time point compared to the bodily fluid at the first time point is indicates progression eczema.

In another aspect, the disclosure contemplates a method for monitoring the efficacy of a treatment for eczema in a subject. In certain embodiments, such method includes a step of contacting a bodily fluid obtained from the subject at a first time point with a plurality of distinct food preparations (e.g., eczema trigger food preparations) coupled to a solid carrier, wherein the contacting is performed under conditions that allow antibodies from the bodily fluid to bind to each distinct food preparation (e.g., eczema trigger food preparations) in the plurality. In other embodiments, the method includes a step of detecting the antibodies bound to the each distinct food preparation (e.g., eczema trigger food preparation) in the plurality at the first time point to obtain an immunoassay signal for each distinct food preparation (e.g., eczema trigger food preparation) in the plurality. In other embodiments, the method includes a step of administering a treatment regimen. In other embodiments, the method includes a step of contacting a bodily fluid obtained from the subject at a second time point with a plurality of distinct food preparations (e.g., eczema trigger food preparations) coupled to a solid carrier, wherein the contacting is performed under conditions that allow antibodies from the bodily fluid to bind to each distinct food preparation (e.g., eczema trigger food preparation) in the plurality. In other embodiments, the method includes a step of detecting the antibodies bound to the each distinct food preparation (e.g., eczema trigger food preparation) in the plurality at the second time point to obtain an immunoassay signal for each distinct eczema trigger food preparation in the plurality. In other embodiments, the method includes a step of comparing the immunoassay signal for each distinct food preparation (e.g., eczema trigger food preparation) in the plurality at the first time point and the second time point, wherein a decreased amount of at least immunoassay signal present in the bodily fluid obtained at the second time point compared to the bodily fluid at the first time point is indicative of an effective treatment for eczema.

In another aspect, the disclosure contemplates a method of generating a test for identifying food items that trigger and/or exacerbate eczema in patients diagnosed with or suspected of having eczema. Thus, in certain embodiments, the method is for identifying triggering food items among already diagnosed or suspected eczema patients. Such test will typically include a step of obtaining test results (e.g., immunoassay) for various distinct food preparations, wherein the test results are based on bodily fluids (e.g., including, but not limited to, whole blood, plasma, serum, saliva, urine, fecal suspension) of patients diagnosed with or suspected to have eczema and bodily fluids of a control group not diagnosed with or not suspected to have eczema. In other embodiments, the method is for identifying triggering food items among patients only suspected of having eczema. In certain embodiments, the test results are then stratified by gender for each of the distinct food preparations, a different cutoff value for male and female patients for each of the distinct food preparations (e.g., cutoff value for male and female patients has a difference of at least 10% (abs)) is assigned for a predetermined percentile rank (e.g., 90th or 95th percentile).

As noted herein, and while not limiting to the inventive subject matter, it is contemplated that in certain embodiments, the plurality of distinct food preparations includes at least two (food preparations prepared from food items selected from foods of Table 1. Regardless of the particular choice of food items, in certain embodiments, the plurality of distinct food preparations has a raw p-value (or an average raw p-value) of ≤0.07 (or ≤0.05, or ≤0.025) or an FDR multiplicity adjusted p-value (or an average FDR multiplicity adjusted p-value) of ≤0.10 (or ≤0.08, or ≤0.07). In other embodiments, the plurality of distinct food preparations has a raw p-value (or an average raw p-value) selected from the group consisting of about ≤0.07, about ≤0.065, about ≤0.06, about ≤0.055, about ≤0.05, about ≤0.045, about ≤0.04, about ≤0.035, about ≤0.03, about ≤0.025, and about ≤0.02. In yet other embodiments, the plurality of distinct food preparations has a FDR multiplicity adjusted p-value (or an average FDR multiplicity adjusted p-value) selected from the group consisting of about ≤0.10, about ≤0.095, about ≤0.09, about ≤0.085, about ≤0.08, about ≤0.075, about ≤0.07, about ≤0.065, about ≤0.06, about K 0.055, and about ≤0.05. Exemplary aspects and protocols, and considerations are provided in the experimental description below.

Thus, it should be appreciated that by having a high-confidence test system as described herein, the rate of false-positive and false negatives can be significantly reduced. Such advantages have heretofore not been realized and it is expected that the test panels, test kits, arrays, etc. and methods presented herein will substantially increase the predictive power of food sensitivity tests for patients diagnosed with or suspected of having eczema.

### EXAMPLES

Example 1: General protocol for food preparation generation: Commercially available food extracts (available from Biomerica Inc., 17571 Von Karman Ave, Irvine, CA 92614) prepared from the edible portion of the respective raw foods were used to prepare the compositions described herein, following the manufacturer's instructions.

For some food extracts, food extracts prepared with certain specific procedures to generate the food extracts, provide superior results in detecting elevated immunoglobulin (e.g., IgG) reactivity in eczema patients compared to other commercially available food extracts. For example, in certain embodiments related to grains and nuts, a three-step procedure of generating food extracts may be used. The first step is a defatting step. In this step, lipids from grains and nuts are extracted by contacting the flour of grains and nuts with a non-polar solvent and collecting residue. Then, the defatted grain or nut flour are extracted by contacting the flour with elevated pH to obtain a mixture and removing the solid from the mixture to obtain a liquid extract. Once the liquid extract is generated, the liquid extract is stabilized by adding an aqueous formulation. In certain embodiments, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

In another example, in certain embodiments related to meats and fish, a two-step procedure of generating food extracts may be used. The first step is an extraction step. In this step, extracts from raw, uncooked meats or fish are generated by emulsifying the raw, uncooked meats or fish in an aqueous buffer formulation in a high impact pressure processor. Then, solid materials are removed to obtain a liquid extract. Once the liquid extract is generated, the liquid extract is stabilized by adding an aqueous formulation. In certain embodiments, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

In yet another example, in certain embodiments related to fruits and vegetables, a two-step procedure of generating food extracts may be used. The first step is an extraction step. In this step, liquid extracts from fruits or vegetables are generated using an extractor (e.g., masticating juicer, etc.) to pulverize foods and extract juice. Then, solid materials are removed to obtain a liquid extract. Once the liquid extract is generated, the liquid extract is stabilized by adding an aqueous formulation. In certain embodiments, the aqueous formulation includes a sugar alcohol, a metal chelating agent, protease inhibitor, mineral salt, and buffer component 20-50 mM of buffer from 4-9 pH. This formulation allowed for long term storage at −70° C. and multiple freeze-thaws without a loss of activity.

Example 2: Blocking of solid support (e.g., ELISA) carrier: To optimize signal to noise, solid support plates were blocked with a blocking buffer. In one embodiment, the blocking buffer includes 20-50 mM of a phosphate buffer (pH 4-9), bovine serum albumin (BSA) and a polyvinyl alcohol (PVA).

Example 3: Solid support (e.g., ELISA) carrier preparation and sample testing: In certain embodiments, food preparations were immobilized onto a respective solid support following the manufacturer's instructions. For the methods described herein, the food preparations immobilized to the solid support were allowed to react with immunoglobulins (antibodies) present in the subject' serum (e.g., IgG antibodies), and the excess serum proteins were removed by a wash step. In certain embodiments, for detection of immunoglobulin (e.g., IgG antibody) binding, enzyme labeled anti-IgG antibody conjugate was allowed to react with food preparation-antibody complex. A color was developed by the addition of a substrate that reacts with the coupled enzyme. The color intensity was measured and is directly proportional to the concentration of IgG antibody specific to a particular food antigen.

Example 4: Methodology to determine ranked food list in order of ability of immunoassay (e.g., ELISA) signals to distinguish eczema from control subjects: In some embodiments, out of an initial selection (e.g., 100 food items, or 150 food items, or even more), food preparations may be eliminated prior to analysis due to low consumption in an intended, or target population. In addition, in other embodiments, the specific food preparations can be used as being representative of the larger more generic food group, especially where prior testing has established a correlation among different species within a generic group (e.g. in both genders, but also suitable for correlation for a single gender). For example, in one embodiment, green pepper could be dropped in favor of chili pepper as representative of the “pepper” food group, or sweet potato could be dropped in favor of potato as representative of the “potato” food group. In other embodiments, the final list foods will be less than 50 food items. In another embodiment, the final list of foods will be equal to or less than of 40 food items. In another embodiment, the final list of foods will be equal to or less than of 30 food items. In another embodiment, the final list of foods will be equal to or less than of 20 food items. In another embodiment, the final list of foods will be equal to or less than of 10 food items. In other embodiments, the final list of foods is selected from the group consisting of less than 50 food items, less than 49 food items, less than 48 food items, less than 47 food items, less than 46 food items, less than 45 food items, less than 44 food items, less than 43 food items, less than 42 food items, less than 41 food items, less than 40 food items, less than 39 food items, less than 38 food items, less than 37 food items, less than 36 food items, less than 35 food items, less than 34 food items, less than 33 food items, less than 32 food items, less than 31 food items, less than 30 food items, less than 29 food items, less than 28 food items, less than 27 food items, less than 26 food items, less than 25 food items, less than 24 food items, less than 23 food items, less than 22 food items, less than 21 food items, less than 20 food items less than 19 food items, less than 18 food items, less than 17 food items, less than 16 food items, less than 15 food items, less than 14 food items, less than 13 food items, less than 12 food items, less than 11 food items, less than 10 food items, less than 9 food items, less than 8 food items, less than 7 food items, less than 6 food items, less than 5 food items, less than 4 food items, less than 3 food items, and less than 2 food items.

In some embodiments, since the foods ultimately selected for a test panel, a test kit, array, etc., as described herein, will not be specific for a particular gender, a gender-neutral food list is necessary. Since the observed sample will be at least initially imbalanced by gender, differences in ELISA signal magnitude strictly due to gender is removed by modeling signal scores against gender using a two-sample t-test and storing the residuals for further analysis. For each of the tested foods (i.e., food preparations), residual signal scores are compared between eczema subjects and (healthy) control subjects using a permutation test on a two-sample t-test with a relative high number of resamplings (e.g., in certain embodiments >1,000 resamplings; in other embodiments >10,000 resamplings; in yet other embodiments >50,000 resamplings). The Satterthwaite approximation is in certain embodiments, then used for the denominator degrees of freedom to account for lack of homogeneity of variances, and the 2-tailed permuted p-value represent the raw p-value for each food. False Discovery Rates (FDR) among the comparisons, is adjusted by any acceptable statistical procedures (e.g., Benjamini-Hochberg, Family-wise Error Rate (FWER), Per Comparison Error Rate (PCER), etc.).

Foods (i.e., food preparations) were then ranked according to their 2-tailed FDR multiplicity-adjusted p-values. Foods with adjusted p-values equal to or lower than the desired FDR threshold are deemed to have significantly higher signal scores among eczema subjects than control subjects and therefore deemed candidates for inclusion into a food panel.

Based on earlier experiments, the inventors contemplate that even for the same food preparation tested, the ELISA score for at least several food items (i.e., food preparations) may vary. As should be readily appreciated, data unstratified by gender may therefore lose significant explanatory power where the same cutoff value is applied to raw data for male and female data. To overcome this, the inventors therefore contemplate in certain embodiments, the stratification of the data by gender as described below.

Example 5: Statistical Method for Cutpoint Selection for each Food: The determination of what immunoassay (e.g., ELISA) signal scores would constitute a “positive” response can be made by summarizing the distribution of signal scores among the Control subjects. For each food (i.e., food preparations), subjects known to have or suspected of having eczema who have observed scores greater than or equal to selected quantiles of the Control subject distribution will be deemed “positive”. To attenuate the influence of any one subject on cutpoint determination, each food-specific (and gender-specific) dataset will be bootstrap resampled 1000 times. Within each bootstrap replicate, the 90th and 95th percentiles of the Control signal scores will be determined. Each subject known to have or suspected of having eczema in the bootstrap sample will be compared to the 90^{th} and 95^{th} percentiles to determine whether he/she had a “positive” response. The final 90^{th} and 95^{th} percentile-based cutpoints for each food (and gender) will be computed as the average 90^{th} and 95^{th} percentiles across the 1000 samples. The number of foods for which each subject known to have or suspected of having eczema will be rated as “positive” was computed by pooling data across foods. Using such method, the inventors can to identify cutoff values for a predetermined percentile rank that in most cases was substantially different.

It should be noted that nothing in the art has provided any predictable food groups related to eczema, moreover predictable food groups related to eczema that are gender-stratified. Thus, a discovery of food items (i.e., food preparations) that show distinct responses by gender is also a surprising result, which could not be obviously expected in view of all previously available art. In other words, selection of food items based on gender stratification provides an unexpected technical effect such that statistical significances for particular food items as triggering food among male or female eczema patients have been significantly improved.

Example 6: Normalization of IgG Response Data: While the raw data of the patient's IgG response results can be used to compare strength of response among given foods, it is also contemplated that the IgG response results of a patient are normalized and indexed to generate unit-less numbers for comparison of relative strength of response to a given food. For example, one or more of a patient's food specific IgG results (e.g., IgG specific to food A or IgG specific to food B) can be normalized to the patient's total IgG. The normalized value of the patient's IgG specific to food A can be 0.1 and the normalized value of the patient's IgG specific to food B can be 0.3. In this scenario, the relative strength of the patient's response to food B is three times higher compared to food A. Then, the patient's sensitivity to food A and food B can be indexed as such.

In other examples, one or more of a patient's food specific IgG results can be normalized to the global mean of that patient's food specific IgG results. The global means of the patient's food specific IgG can be measured by total amount of the patient's food specific IgG. In this scenario, the patient's specific IgG to food A can be normalized to the mean of patient's total food specific IgG (e.g., mean of IgG levels to food A, food B, food C, food D, food E, etc.). However, it is also contemplated that the global means of the patient's food specific IgG can be measured by the patient's IgG levels to a specific type of food via multiple tests. If the patient has been tested for his sensitivity to food A five times and to food B seven times previously, the patient's new IgG values to food A or to food B are normalized to the mean of five-times test results to food A or the mean of seven-times test results to food B. The normalized value of the patient's IgG specific to food A can be 6.0 and the normalized value of the patient's IgG specific to food B can be 1.0. In this scenario, the patient has six times higher sensitivity to food A at this time compared to his average sensitivity to food A, but substantially similar sensitivity to food B. Then, the patient's sensitivity to food A and food B can be indexed based on such comparison.

Example 7: Methodology to determine the subset of eczema patients with food sensitivities that underlie eczema: While it is suspected that food sensitivities play a substantial role in signs and symptoms of eczema, some eczema patients may not have food sensitivities that underlie eczema. Those patients would not be benefit from dietary intervention to treat signs and symptoms of eczema. To determine the subset of such patients, body fluid samples of eczema patients and non-eczema patients can be tested using the compositions and methods described herein.

Average and median number of positive foods will be computed for eczema and non-eczema “healthy” control patients. Using these raw data, average and standard deviation of the number of positive foods (i.e., trigger foods) will be computed for eczema and non-eczema patients. Thus, it can be appreciated that an eczema patient having sensitivity to zero positive foods is unlikely to have food sensitivities underlying their signs and symptoms of eczema.

The statistical data includes normality, arithmetic mean, median, percentiles and 95% confidence interval (CI) for the mean and median representing number of positive foods (i.e., trigger foods) in the eczema population and the non-eczema population. These raw data will be transformed by logarithmic transformation to improve the data interpretation.

The statistical data of an independent T-test (logarithmically transformed data) and a Mann-Whitney test will be used to compare the geometric mean number of positive foods (i.e., trigger foods) between the eczema and non-eczema samples. The data shown will indicate statistically significant differences in the geometric mean of positive number of foods between the eczema population and the non-eczema population.

The disclosure uses exemplary statistical data of a Receiver Operating Characteristic (ROC) curve analysis of data to determine the diagnostic power of the test at discriminating eczema from non-eczema subjects. The p-value for the ROC is significant at a p-value of ≤0.0001. Because the statistical difference between the eczema population and the non-eczema population is expected to be significant when the test results are cut off to a positive number of, for example, four, the number of foods for which a patient tests positive could be used as a confirmation of the primary clinical diagnosis of eczema, and whether it is likely that food sensitivities underlies on the patient's signs and symptoms of eczema. Therefore, the above test can be used as another ‘rule in’ test to add to currently available clinical criteria for diagnosis for eczema.

Example 8: Method for determining distribution of per-person number of foods declared “positive”: To determine the distribution of number of “positive” foods (i.e., trigger foods) per person and measure the diagnostic performance, the analysis will be performed with a group of 40 food preparations (or, e.g., 20, or 30, or 50 food preparations) from Table 1, which showed the most positive responses to eczema patients. To attenuate the influence of any one subject on this analysis, each food-specific and gender-specific dataset will be bootstrap resampled 1000 times. Then, for each food item in the bootstrap sample, sex-specific cutpoint will be determined using the 90^{th} and 95^{th} percentiles of the control population. Once the sex-specific cutpoints are determined, the sex-specific cutpoints will be compared with the observed ELISA signal scores for both control and eczema subjects. In this comparison, if the observed signal is equal or more than the cutpoint value, then it will be determined “positive” food, and if the observed signal is less than the cutpoint value, then it will be determined “negative” food.

Once all food items (i.e., food preparations) were determined either positive or negative, the results of the, e.g., 80 (40 foods×2 cutpoints) calls for each subject were saved within each bootstrap replicate. Then, for each subject, 40 calls were summed using 90^{th} percentile as cutpoint to get “Number of Positive Foods (90^{th}),” and the rest of 40 calls will be summed using 95^{th} percentile to get “Number of Positive Foods (95^{th}).” Then, within each replicate, “Number of Positive Foods (90^{th})” and “Number of Positive Foods (95^{th})” were summarized across subjects to get descriptive statistics for each replicate as follows: 1) overall means equals to the mean of means, 2) overall standard deviation equals to the mean of standard deviations, 3) overall medial equals to the mean of medians, 4) overall minimum equals to the minimum of minimums, and 5) overall maximum equals to maximum of maximum. In this analysis, to avoid non-integer “Number of Positive Foods” when computing frequency distribution and histogram, the inventors will assume that the 1000 repetitions of the same original dataset were actually 999 sets of new subjects of the same size added to the original sample. Once the summarization of data is done, frequency distributions and histograms will be generated for both “Number of Positive Foods (90^{th})” and “Number of Positive Foods (95^{th})” for both genders and for both eczema subjects and control subjects using programs “a_pos_foods.sas, a_pos_foods_by_dx.sas”.

Example 9: Method for measuring diagnostic performance: To measure diagnostic performance for each food items (i.e., food preparations) for each subject, data will be used of “Number of Positive Foods (90^{th})” and “Number of Positive Foods (95^{th})” for each subject within each bootstrap replicate described above. In this analysis, the cutpoint was set to 1. Thus, if a subject has one or more “Number of Positive Foods (90^{th})”, then the subject will be called “Has Eczema.” If a subject has less than one “Number of Positive Foods (90^{th})”, then the subject will be called “Does Not Have Eczema.” When all calls are made, the calls will be compared with actual diagnosis to determine whether a call was a True Positive (TP), True Negative (TN), False Positive (FP), or False Negative (FN). The comparisons will be summarized across subjects to get the performance metrics of sensitivity, specificity, positive predictive value, and negative predictive value for both “Number of Positive Foods (90^{th})” and “Number of Positive Foods (95^{th})” when the cutpoint is set to 1 for each method. Each (sensitivity, 1-specificity) pair becomes a point on the ROC curve for this replicate.

To increase the accuracy, the analysis above was repeated by incrementing cutpoint from 2 up to 40, and repeated for each of the 1000 bootstrap replicates. Then the performance metrics across the 1000 bootstrap replicates will be summarized by calculating averages using a program “t_pos_foods_by_dx.sas”.

Of course, it should be appreciated that certain variations in the food preparations may be made without altering the inventive subject matter presented herein. For example, where the food item was yellow onion, that item should be understood to also include other onion varieties that were demonstrated to have equivalent activity in the tests. Indeed, the inventors have noted that for each tested food preparation, certain other related food preparations also tested in the same or equivalent manner (data not shown). Thus, it should be appreciated that each tested and claimed food preparation will have equivalent related preparations with demonstrated equal or equivalent reactions in the test.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

TABLE 1

Abalone
Adlay
Almond
American Cheese
Apple
Artichoke
Asparagus
Avocado
Baby Bok Choy
Bamboo shoots
Banana
Barley, whole grain
Beef
Beets
Beta-lactoglobulin
Blueberry
Broccoli
Buckwheat
Butter
Cabbage
Cane sugar
Cantaloupe
Caraway
Carrot
Casein
Cashew
Cauliflower
Celery
Chard
Cheddar Cheese
Chick Peas
Chicken
Chili pepper
Chocolate
Cinnamon
Clam
Cocoa Bean
Coconut
Codfish
Coffee
Cola nut
Corn
Cottage cheese
Cow's milk
Crab
Cucumber
Cured Cheese
Cuttlefish
Duck
Durian
Eel
Egg White (separate)
Egg Yolk (separate)
Egg, white/yolk (comb.)
Eggplant
Garlic
Ginger
Gluten - Gliadin
Goat's milk
Grape, white/concord
Grapefruit
Grass Carp
Green Onion
Green pea
Green pepper
Guava
Hair Tail
Hake
Halibut
Hazelnut
Honey
Kelp
Kidney bean
Kiwi Fruit
Lamb
Leek
Lemon
Lentils
Lettuce, Iceberg
Lima bean
Lobster
Longan
Mackerel
Malt
Mango
Marjoram
Millet
Mung bean
Mushroom
Mustard seed
Oat
Olive
Onion
Orange
Oyster
Papaya
Paprika
Parsley
Peach
Peanut
Pear
Pepper, Black
Pineapple
Pinto bean
Plum
Pork
Potato
Rabbit
Rice
Roquefort Cheese
Rye
Saccharine
Safflower seed
Salmon
Sardine
Scallop
Sesame
Shark fin
Sheep's milk
Shrimp
Sole
Soybean
Spinach
Squashes
Squid
Strawberry
String bean
Sunflower seed
Sweet potato
Swiss cheese
Taro
Tea, black
Tobacco
Tomato
Trout
Tuna
Turkey
Vanilla
Walnut, black
Watermelon
Welch Onion
Wheat
Wheat bran
Yeast (S. cerevisiae)
Yogurt
FOOD ADDITIVES
Arabic Gum
Carboxymethyl Cellulose
Carrageneenan
FD&C Blue #1
FD&C Red #3
FD&C Red #40
FD&C Yellow #5
FD&C Yellow #6
Gelatin
Guar Gum
Maltodextrin
Pectin
Whey
Xanthan Gum

What is claimed is:

1. A test panel for testing food sensitivity in a human subject diagnosed with or suspected of having eczema, comprising:
one or more distinct food preparations, wherein each distinct food preparation is independently coupled to a spatially addressable solid carrier;
wherein at least 70% of the one or more distinct food preparations have a raw p-value of ≤0.07 or a false discovery rate (FDR) multiplicity adjusted p-value of ≤0.10.

2. A test panel for testing food sensitivity in a human subject diagnosed with or suspected of having eczema, consisting essentially of:
one or more distinct food preparations, wherein each distinct food preparation is independently coupled to a spatially addressable solid carrier;
wherein the one or more distinct food preparations have a raw p-value of ≤0.07 or a false discovery rate (FDR) multiplicity adjusted p-value of ≤0.10.

3. The test panel of claim 1 or 2, wherein the one or more distinct food preparations includes at least 4 food preparations selected from foods in Table 1.

4. The test panel of claim 1 or 2, wherein the one or more distinct food preparations includes at least 5 food preparations selected from foods in Table 1.

5. The test panel of claim 1 or 2, wherein the one or more distinct food preparations includes at least 6 food preparations selected from foods in Table 1.

6. The test panel of claim 1 or 2, wherein the one or more distinct food preparations includes at least 8 food preparations selected from foods in Table 1.

7. The test panel of claim 1 or 2, wherein the one or more distinct food preparations includes at least 10 food preparations prepared from food items of Table 1.

8. The test panel of claim 1 or 2, wherein the one or more distinct food preparations includes at least 12 food preparations prepared from food items of Table 1.

9. The test panel of any one of claims 1-8, wherein the one or more distinct food preparations has a raw p-value of ≤0.05 or a FDR multiplicity adjusted p-value of ≤0.08.

10. The test panel of any one of claims 1-8, wherein the one or more distinct food preparations has a raw p-value of ≤0.025 or a FDR multiplicity adjusted p-value of ≤0.07.

11. The test panel of claim 1, wherein at least 75% of the one or more distinct food preparations have a raw p-value of ≤0.07 or FDR multiplicity adjusted p-value of ≤0.10.

12. The test panel of claim 1, wherein at least 80% of the one or more distinct food preparations have a raw p-value of ≤0.07 or FDR multiplicity adjusted p-value of ≤0.10.

13. The test panel of claim 1, wherein at least 85% of the one or more distinct food preparations have a raw p-value of ≤0.07 or FDR multiplicity adjusted p-value of ≤0.10.

14. The test panel of claim 1, wherein at least 90% of the one or more distinct food preparations have a raw p-value of ≤0.07 or FDR multiplicity adjusted p-value of ≤0.10.

15. The test panel of claim 1, wherein at least 95% of the one or more distinct food preparations have a raw p-value of ≤0.07 or FDR multiplicity adjusted p-value of ≤0.10.

16. The test panel of any one of claims 1-15, wherein FDR multiplicity adjusted p-value is adjusted for age or gender.

17. The test panel of any one of claims 1-15, wherein FDR multiplicity adjusted p-value is adjusted for age and gender.

18. The test panel of any one of the claims 1-17, wherein the one or more distinct food preparations comprise crude filtered aqueous extracts.

19. The test panel of any one of the claims 1-17 wherein the one or more distinct food preparations comprise processed aqueous extracts.

20. The test panel of any one of the claims 1-17 wherein the one or more distinct food preparations comprise crude filtered aqueous extracts or processed aqueous extracts.

21. The test panel of any one of claims 1-20, wherein the p-value is determined by a process comprising comparing assay values of a first subject test cohort that is diagnosed with or is suspected of having eczema with assay values of a second subject test cohort that is not diagnosed with or is not suspected of having eczema.

22. The test panel of any one of claims 1-20, wherein the p-value is determined by a process comprising comparing a mean or median immunoglobulin immunoassay score for a distinct food preparation from a first subject test group that is diagnosed with or is suspected of having eczema to at least the 90^{th} percentile rank of a mean or median immunoglobulin immunoassay score for the distinct food preparation from a second subject test group that is not diagnosed with or is not suspected of having eczema.

23. The test panel of any one of claims 1-20, wherein the p-value is determined by a process comprising comparing a mean or median immunoglobulin immunoassay score for a distinct food preparation from a first subject test group that is diagnosed with or is suspected of having eczema to at least the 95^{th} percentile rank of a mean or median immunoglobulin immunoassay score for the distinct food preparation from a second subject test group that is not diagnosed with or is not suspected of having eczema.

24. The test panel of claim 22 or 23, wherein the immunoassay is an ELISA or an antibody capture enzyme immunoassay.

25. The test panel of any one of claims 1-24, wherein the solid carrier is an array, a multi-well plate, a microtiter plate, a microchip, a bead, a disc, a sensor, an adsorptive film, a membrane, a glass slide, a magnetic particle, a dipstick, or a microfluidic device.

26. The test panel of claim 25, wherein the array is a spotted microarray or a lateral flow array.

27. The test panel of claim 25, wherein the sensor is an electrical sensor, a chemical sensor, or a fiber optic sensor.

28. The test panel of claim 25, wherein the membrane is a nitrocellulose membrane, a polytetrafluorethylene membrane, a cellulose nitrate membrane or a cellulose acetate membrane.

29. The test panel of claim 1 or 2, wherein all of the one or more distinct food preparations have an average raw p-value of ≤0.07 or an average false discovery rate (FDR) multiplicity adjusted p-value of ≤0.10.

30. The test panel of any one of claims 1-29, wherein the FDR multiplicity adjusted p-value is adjusted for age and/or gender.

31. A test kit comprising the test panel of any one of claims 1-30.

32. An eczema test panel for the simultaneous detection of one or more eczema trigger foods, comprising:
a plurality of distinct eczema trigger food preparations, wherein each distinct eczema trigger food preparation is independently immobilized to a spatially addressable solid carrier;
wherein at least 70% of the plurality of distinct eczema trigger food preparations each have a raw p-value of ≤0.07 or a FDR multiplicity adjusted p-value of ≤0.10.

33. An eczema test panel for the simultaneous detection of one or more eczema trigger foods, consisting essentially of:
a plurality of distinct eczema trigger food preparations, wherein each distinct eczema trigger food preparation is independently immobilized to a spatially addressable solid carrier;
wherein the plurality of distinct eczema trigger food preparations each have a raw p-value of ≤0.07 or a FDR multiplicity adjusted p-value of ≤0.10.

34. The eczema test panel of claim 32 or 33, wherein the plurality of distinct eczema trigger food preparations includes at least 4 distinct eczema trigger food preparations.

35. The eczema test panel of claim 32 or 33, wherein the plurality of distinct eczema trigger food preparations includes at least 5 distinct eczema trigger food preparations.

36. The eczema test panel of claim 32 or 33, wherein the plurality of distinct eczema trigger food preparations includes at least 6 distinct eczema trigger food preparations.

37. The eczema test panel of claim 32 or 33, wherein the plurality of distinct eczema trigger food preparations includes at least 7 distinct eczema trigger food preparations.

38. The eczema test panel of claim 32 or 33, wherein the plurality of distinct eczema trigger food preparations includes at least 8 distinct eczema trigger food preparations.

39. The eczema test panel of claim 32 or 33, wherein the plurality of distinct eczema trigger food preparations includes at least 9 distinct eczema trigger food preparations.

40. The eczema test panel of claim 32 or 33, wherein the plurality of distinct eczema trigger food preparations includes at least 10 distinct eczema trigger food preparations.

41. The eczema test panel of claim 32 or 33, wherein the plurality of distinct eczema trigger food preparations includes at least 11 distinct eczema trigger food preparations.

42. The eczema test panel of claim 32 or 33, wherein the plurality of distinct eczema trigger food preparations includes at least 12 distinct eczema trigger food preparations.

43. The eczema test panel of claim any one of claims 32-42, wherein the plurality of distinct eczema trigger food preparations each have a raw p-value of ≤0.05 or FDR multiplicity adjusted p-value of ≤0.08.

44. The eczema test panel of claim any one of claims 32-42, wherein the plurality of distinct eczema trigger food preparations each have a raw p-value of ≤0.025 or FDR multiplicity adjusted p-value of ≤0.07.

45. The eczema test panel of any one of claims 32-44, wherein FDR multiplicity adjusted p-value is adjusted for age or gender.

46. The eczema test panel of any one of claims 32-44, wherein FDR multiplicity adjusted p-value is adjusted for age and gender.

47. The eczema test panel of any one of the claims 32-45, wherein the plurality of distinct eczema trigger food preparations comprises crude filtered aqueous extracts.

48. The eczema test panel of any one of the claims 32-45, wherein the plurality of distinct eczema trigger food preparations comprises processed aqueous extracts.

49. The eczema test panel of any one of the claims 32-45, wherein the plurality of distinct eczema trigger food preparations comprises crude filtered aqueous extracts or processed aqueous extracts.

50. The eczema test panel of any one of claims 32-45, wherein the p-value is determined by a process comprising comparing assay values of a first subject test cohort that is diagnosed with or is suspected of having eczema with assay values of a second subject test cohort that is not diagnosed with or is not suspected of having eczema.

51. The eczema test panel of any one of claims 32-45, wherein the p-value is determined by a process comprising comparing a mean or median immunoglobulin immunoassay scores for a distinct food preparation from a first subject test group that is diagnosed with or is suspected of having eczema to at least the 90^{th} percentile rank of the mean or median immunoglobulin immunoassay scores for the distinct food preparation from a second subject test group that is not diagnosed with or is not suspected of having eczema.

52. The eczema test panel of any one of claims 32-45, wherein the p-value is determined by a process comprising comparing a mean or median immunoglobulin immunoassay scores for a distinct food preparation from a first subject test group that is diagnosed with or is suspected of having eczema to at least the 95^{th} percentile rank of the mean or median immunoglobulin immunoassay scores for the distinct food preparation from a second subject test group that is not diagnosed with or is not suspected of having eczema.

53. The eczema test panel of claim 51 or 52, wherein the immunoassay is an ELISA or an antibody capture enzyme immunoassay.

54. The eczema test panel of any one of claims 32-53, wherein the solid carrier is an array, a multi-well plate, a microtiter plate, a microchip, a bead, a disc, a sensor, an adsorptive film, a membrane, a glass slide, a magnetic particle, a dipstick, or a microfluidic device.

55. The test panel of claim 54, wherein the array is a spotted microarray or a lateral flow array.

56. The test panel of claim 54, wherein the sensor is an electrical sensor, a chemical sensor, or a fiber optic sensor.

57. The test panel of claim 54, wherein the membrane is a nitrocellulose membrane, a polytetrafluorethylene membrane, a cellulose nitrate membrane or a cellulose acetate membrane.

58. The test panel of claim 32, wherein the totality of the plurality of distinct eczema trigger food preparations has an average raw p-value of ≤0.07 or an average false discovery rate (FDR) multiplicity adjusted p-value of ≤0.10.

59. The test panel of any one of claims 32-58, wherein the FDR multiplicity adjusted p-value is adjusted for age and/or gender.

60. A test kit comprising the eczema test panel of any one of claims 32-59.

61. A method of identifying one or more distinct food preparations for a subject known to have or is suspected of having eczema, the method comprising:
contacting a bodily fluid of a subject that is known to have or is suspected of having eczema with a plurality of distinct food preparations coupled to a solid carrier, wherein the contacting is performed under conditions that allow antibodies from the bodily fluid to bind to a first distinct food preparation,
detecting the antibodies bound to the first distinct food preparation to obtain a first immunoassay signal;
comparing the first immunoassay signal to a first control signal, and
identifying one or more distinct food preparations positive for the subject known to have or is suspected of having eczema.

62. A method of identifying a human subject in need of medical treatment for eczema, the method comprising:
obtaining a bodily fluid from the subject,
contacting the bodily fluid of the subject with a plurality of distinct food preparations coupled to a solid carrier, wherein the contacting is performed under conditions that allow antibodies from the bodily fluid to bind to each distinct food preparation in the plurality,
detecting the antibodies bound to the each distinct food preparation in the plurality to obtain an immunoassay signal for each distinct food preparation in the plurality;
comparing the immunoassay signal for each distinct food preparation in the plurality to a control signal for each distinct food preparation in the plurality, and
identifying the distinct food preparations positive for the subject, thereby determining the need for medical treatment for the subject.

63. The method of claim 61, wherein the first control signal is determined by assigning a predetermined percentile rank cutoff value for the first distinct food preparation, and wherein the predetermined percentile rank cutoff value is an at least 90^{th} percentile rank of the control signal from a control group or known standard.

64. The method of claim 61, further comprising detecting the antibodies bound to a second distinct food preparation to obtain a second immunoassay signal and comparing the second immunoassay signal to a second control signal.

65. The method of claim 64, wherein the second control signal is determined by assigning a predetermined percentile rank cutoff value for the second distinct food preparation, and wherein the predetermined percentile rank cutoff value is an at least 90^{th} percentile rank of the second control signal from a control group or known standard.

66. The method of claim 61, further comprising detecting the antibodies bound to each of the plurality of distinct food preparations to obtain an immunoassay signal for each distinct food preparation and comparing each immunoassay signal to a control signal for each distinct food preparation.

67. The method of claim 66, wherein the control signal for each distinct food preparation is determined by assigning a predetermined percentile rank cutoff value for each distinct food preparation, and wherein the predetermined percentile rank cutoff value is an at least 90^{th} percentile rank of the second control signal from a control group or known standard.

68. The method of claim 62, wherein the control signal for each distinct food preparation is determined by assigning a predetermined percentile rank cutoff value for each distinct food preparation, and wherein the predetermined percentile rank cutoff value is an at least 90^{th} percentile rank of the control signal from a control group or known standard.

69. The method of any one of claims 61-68, wherein the detecting comprises measuring or quantifying the antibodies via an immunoassay.

70. The method of claim 61, wherein the first control signal is determined by contacting a bodily fluid of a non-eczema healthy control subject with a first distinct food preparation coupled to a solid carrier, wherein the contacting is performed under conditions that allow antibodies from the bodily fluid to bind to at least one component of a distinct food preparation and measuring or quantifying the antibodies via an immunoassay.

71. The method of claim 69 or 70, wherein the immunoassay is an ELISA or an antibody capture enzyme immunoassay.

72. The method of claim 61, wherein the first control signal is determined by comparing the first immunoassay signal against a gender-stratified reference value for the distinct food preparation.

73. The method of claim 62, wherein the control signal is determined by comparing the immunoassay signal for each distinct food preparation in the plurality against a gender-stratified reference value for the distinct food preparation.

74. The method of claim 61, wherein the subject is diagnosed with eczema.

75. The method of claim 61 or 62, further comprising generating a report for the distinct food preparations positive for the subject.

76. The method of claim 61, further comprising generating a report for the one or more distinct food preparations positive for the subject for the potential elimination from the subject's diet.

77. The method of claim 62 or 76, further comprising treating the subject having eczema, wherein the treatment comprises eliminating one or more of the identified eczema trigger foods from the subject's diet.

78. The method of any one of claims 61-77, wherein the plurality of distinct food preparations are food preparations having a raw p-value of ≤0.07 or a false discovery rate (FDR) multiplicity adjusted p-value of ≤0.10.

79. The method of any one of claims 61-77, wherein at least 70% of the plurality of distinct food preparations have a raw p-value of ≤0.07 or FDR multiplicity adjusted p-value of ≤0.10.

80. The method of any one of claims 61-77, wherein the totality of the plurality of distinct food preparations has an average raw p-value of ≤0.07 or an average false discovery rate (FDR) multiplicity adjusted p-value of ≤0.10

81. The method of any one of claims 61-77, wherein the plurality of distinct food preparations are food preparations having a raw p-value of ≤0.05 or FDR multiplicity adjusted p-value of ≤0.08, or a raw p-value of ≤0.025 or FDR multiplicity adjusted p-value of ≤0.07.

82. The method of any one of claims 61-81, wherein the solid carrier includes four or more distinct food preparations.

83. The method of any one of claims 61-82, wherein each distinct food preparation is independently immobilized to the solid carrier in a spatially addressable manner.

84. The method of any one of claims 61-83, wherein the solid carrier is an array, a multi-well plate, a microtiter plate, a microchip, a bead, a disc, a sensor, an adsorptive film, a membrane, a glass slide, a magnetic particle, a dipstick, or a microfluidic device.

85. The method of claim 84, wherein the array is a spotted microarray or a lateral flow array.

86. The method of claim 84, wherein the sensor is an electrical sensor, a chemical sensor, or a fiber optic sensor.

87. The method of claim 84, wherein the membrane is a nitrocellulose membrane, a polytetrafluorethylene membrane, a cellulose nitrate membrane or a cellulose acetate membrane.

88. The method of any one of claims 61-87, wherein the antibody has an isotype selected from the group consisting of IgG, IgA, IgM, and IgD.

89. The method of any one of claims 61-87, wherein the antibody is not an IgE.

90. The method of any one of claims 61-89, wherein the bodily fluid is whole blood, plasma, serum, saliva, urine or a fecal suspension.

91. The method of any one of claims 61-90, wherein FDR multiplicity adjusted p-value is adjusted for age and/or gender.

92. The method of any one of claims 61-91, wherein the one or more distinct food preparations comprises crude filtered aqueous extracts and/or processed aqueous extracts.

93. A method of reducing or relieving eczema symptoms in a human subject known to have or suspected of having eczema, the method comprising:
identifying one or more distinct food preparations that are positive for a subject known to have or is suspected of having eczema according to the method of any one of claims 61, 63-67, 70, 72, 74 or 76, wherein eczema symptoms are reduced or alleviated in the subject by eliminating one or more of the identified eczema trigger foods from the subject's diet.

94. A method for monitoring the progression of eczema in a human subject, the method comprising
contacting a bodily fluid obtained from the subject at a first time point with a plurality of distinct food preparations coupled to a solid carrier, wherein the contacting is performed under conditions that allow antibodies from the bodily fluid to bind to each distinct food preparation in the plurality,
detecting the antibodies bound to the each distinct food preparation in the plurality at the first time point to obtain an immunoassay signal for each distinct food preparation in the plurality;
contacting a bodily fluid obtained from the subject at a second time point with a plurality of distinct food preparations coupled to a solid carrier, wherein the contacting is performed under conditions that allow antibodies from the bodily fluid to bind to each distinct food preparation in the plurality,
detecting the antibodies bound to the each distinct food preparation in the plurality at the second time point to obtain an immunoassay signal for each distinct food preparation in the plurality;
comparing the immunoassay signal for each distinct food preparation in the plurality at the first time point and the second time point, wherein an increased amount of at least one immunoassay signal present in the bodily fluid obtained at the second time point compared to the bodily fluid at the first time point is indicates progression of eczema.

95. A method for monitoring the efficacy of a treatment for eczema in a human subject, the method comprising
contacting a bodily fluid obtained from the subject at a first time point with a plurality of distinct food preparations coupled to a solid carrier, wherein the contacting is performed under conditions that allow antibodies from the bodily fluid to bind to each distinct food preparation in the plurality,
detecting the antibodies bound to the each distinct food preparation in the plurality at the first time point to obtain an immunoassay signal for each distinct food preparation in the plurality;
administering a treatment regimen,
contacting a bodily fluid obtained from the subject at a second time point with a plurality of distinct food preparations coupled to a solid carrier, wherein the contacting is performed under conditions that allow antibodies from the bodily fluid to bind to each distinct food preparation in the plurality,
detecting the antibodies bound to the each distinct food preparation in the plurality at the second time point to obtain an immunoassay signal for each distinct food preparation in the plurality;
comparing the immunoassay signal for each distinct food preparation in the plurality at the first time point and the second time point, wherein a decreased amount of at least immunoassay signal present in the bodily fluid obtained at the second time point compared to the bodily fluid at the first time point is indicative of an effective treatment for eczema.

96. The method of claim 94, wherein an increased amount of at least 2, at least 3, at least 4, at least 5, at least 6, at least 7, or at least 8 immunoassay signals present in the bodily fluid obtained at the second time point compared to the bodily fluid at the first time point is indicates progression of eczema.

97. The method of claim 95, wherein a decreased amount of at least 2, at least 3, at least 4, at least 5, at least 6, at least 7, or at least 8 immunoassay signals present in the bodily fluid obtained at the second time point compared to the bodily fluid at the first time point is indicative of an effective treatment for eczema.

98. The method of any one of claims 94-97, wherein the plurality of distinct food preparations are food preparations having a raw p-value of ≤0.07 or a false discovery rate (FDR) multiplicity adjusted p-value of ≤0.10.

99. The method of any one of claims 94-98, wherein the detecting comprises measuring or quantifying the antibodies via an immunoassay.

100. The method of claim 99, wherein the immunoassay is an ELISA or an antibody capture enzyme immunoassay.

101. The method of any one of claims 94-100, further comprising treating the subject, wherein the treatment comprises eliminating one or more of the identified distinct food preparations from the subject's diet.

102. A method of generating a test panel for food sensitivity in a human subject known to have or suspected of having eczema, the method comprising:
obtaining immunoassay results for a plurality of distinct eczema trigger food preparations, wherein the immunoassay results are based on bodily fluids of subjects known to have or suspected of having eczema and bodily fluids of a control group not known to have or not suspected to have eczema;
selecting a plurality of the distinct food preparations, wherein the distinct food preparations are food preparations having a raw p-value of ≤0.07 or a false discovery rate (FDR) multiplicity adjusted p-value of ≤0.10; and
generating a test comprising of the selected distinct food preparations.

103. The method of claim 102, further comprising assigning a predetermined percentile rank cutoff value for each of the plurality of the distinct food preparations, wherein the predetermined percentile rank cutoff value is determined using an at least 90^{th} percentile rank of the immunoassay results based on bodily fluids of the control group.

104. The method of claim 102 or 103, further comprising comparing the immunoassay results for the plurality of distinct food preparations based on bodily fluids of patients known to have or suspected of having eczema to the predetermined percentile rank cutoff value.

105. The method of any one of claims 102-104, further comprising stratifying the immunoassay results by gender for each of the distinct food preparations.

106. The method of any one of claims 102-105, further comprising assigning a predetermined percentile rank a cutoff value for male and female subjects for each of the distinct food preparations.

107. The method of any one of claims 102-106, further comprising normalizing the immunoassay results to the patient's total IgG.

108. The method of any one of claims 102-106, further comprising normalizing the immunoassay results to the global mean of the patient's food specific IgG results.

109. The method of any one of claims 103-107, wherein the predetermined percentile rank is an at least 95^{th} percentile rank.

110. The method of any one of claims 102-109, wherein the plurality of distinct food preparations are food preparations having a raw p-value of ≤0.07 or a false discovery rate (FDR) multiplicity adjusted p-value of ≤0.10.

111. The method of any one of claims 102-110, wherein the totality of the plurality of distinct food preparations has an average raw p-value of ≤0.07 or an average false discovery rate (FDR) multiplicity adjusted p-value of ≤0.10.

112. The method of any one of claims 102-109, wherein the plurality of distinct food preparations are food preparations having a raw p-value of ≤0.05 or FDR multiplicity adjusted p-value of ≤0.08, or a raw p-value of ≤0.025 or FDR multiplicity adjusted p-value of ≤0.07.

113. The method of any one of claims 102-112, wherein the plurality of food preparations is coupled to the solid carrier in a spatially addressable manner.

114. The method of claim 113, wherein the solid carrier includes four or more food preparations.

115. The method of claim 113 or 114, wherein the solid carrier is an array, a multi-well plate, a microtiter plate, a microchip, a bead, a disc, a sensor, an adsorptive film, a membrane, a glass slide, a magnetic particle, a dipstick, or a microfluidic device.

116. The method of claim 115, wherein the array is a spotted microarray or a lateral flow array.

117. The method of claim 115, wherein the sensor is an electrical sensor, a chemical sensor, or a fiber optic sensor.

118. The method of claim 115, wherein the membrane is a nitrocellulose membrane, a polytetrafluorethylene membrane, a cellulose nitrate membrane or a cellulose acetate membrane.

119. The method of any one of claims 102-118, wherein at least 70% of the distinct food preparations have a raw p-value of ≤0.07 or FDR multiplicity adjusted p-value of ≤0.10.

120. The method of any one of claims 102-118, wherein at least 80% of the distinct food preparations have a raw p-value of ≤0.07 or FDR multiplicity adjusted p-value of ≤0.10.

121. The method of any one of claims 102-118, wherein at least 90% of the distinct food preparations have a raw p-value of ≤0.07 or FDR multiplicity adjusted p-value of ≤0.10.

122. The method of any one of claims 102-118, wherein at least 95% of the distinct food preparations have a raw p-value of ≤0.07 or FDR multiplicity adjusted p-value of ≤0.10.

123. The method of any one of claims 102-122, wherein FDR multiplicity adjusted p-value is adjusted for age and/or gender.

124. The method of any one of claims 72-93, wherein the distinct food preparations comprise crude filtered aqueous extracts and/or processed aqueous extracts.

125. A test panel generated according to the method of any one of claims 102-124.

126. A test kit comprising the test panel according to claim 125.
